(19) United States
(12) Patent Application Publication
(10) Pub. No.: US 2013/0337075 A1
(43) Pub. Date: Dec. 19, 2013

(54) NANOMEDICINES FOR EARLY NERVE REPAIR

(71) Applicant: Ji-Xin Cheng, West Lafayette, IN (US)

(72) Inventor: Ji-Xin Cheng, West Lafayette, IN (US)

(73) Assignee: PURDUE RESEARCH FOUNDATION, West Lafayette, IN (US)

(21) Appl. No.: 14/001,189

(22) PCT Filed: Feb. 24, 2012
(86) PCT No.: PCT/US12/26590

§ 371 Date: Aug. 23, 2013

Related U.S. Application Data
(60) Provisional application No. 61/446,252, filed on Feb. 24, 2011.

(51) Int. Cl. A61K 47/48; A61K 45/06

(52) U.S. Cl. CPC A61K 47/4823; A61K 45/06; A61K 47/48884
USPC 424/499; 536/55.1; 514/54

(57) ABSTRACT

The present disclosure describes hydrophobically modified nanoparticles and polymeric nanostructures that can be utilized to for the treatment of neuronal injury or neuronal disease in an affected patient, along with methods of forming and using the nanoparticles and nanostructures. Furthermore, the nanoparticles and nanostructures are designed as “dual action” compositions to treat neuronal injury and neuronal disease via repair of damaged membrane and suppression of intracellular inflammation.

### CROSS REFERENCE TO RELATED APPLICATIONS

This application claims the benefit under 35 USC §119(e) of U.S. Provisional Application Ser. No. 61/446,252 filed on Feb. 24, 2011 the entire disclosure of which is incorporated herein by reference.

### TECHNICAL FIELD

This invention generally pertains to the field of nanomedicine. More particularly, the invention pertains to hydrophobically modified nanoparticles and polymeric nanostructures and methods of forming and using the same.

### BACKGROUND AND SUMMARY OF THE INVENTION

Neural injuries and neural diseases are debilitating and complex manifestations of the body. For example, spinal cord injury (SCI) results in immediate initial disruption of cell membranes in affected neural and endothelial tissues, followed by extensive secondary neurodegenerative processes. Most SCI cases involve a primary injury and a subsequent secondary damage. During the primary injury, the acute mechanical stress to the spinal cord breaks neural membranes and causes Ca²⁺ influx into cells. The latter processes trigger a series of secondary biological events including inflammation, free radical release, and apoptosis, which further exacerbate the damage.

Among various treatments under investigation, a key approach is to seal the damaged membrane at the early stage of SCI. To date, poly(ethylene glycol) (PEG) and Pluronic P188 have been used for membrane repair. However, the effectiveness of these agents has been very limited partly due to their rapid clearance after systemic administration. For example, a PEG without hydrophobic modification can result in negligible efficacy of the treatment of neural injuries.

Applicant has demonstrated a function of block copolymer micelles as a nanoscale membrane repair agent in traumatically injured spinal cord (Shi et al. (2010) Nature Nanotechnology 5:80-87, incorporated herein by reference). Axonal membranes injured by compression may be effectively repaired by self-assembled monomethoxy poly(ethylene glycol)-poly(D,L-lactic acid) (mPEG-PDLLA) di-block copolymer micelles (60 nm in diameter). Intravenously injected mPEG-PDLLA micelles recover locomotor function and reduce the volume and inflammatory response of the lesion in SCI rats. Mechanistically, it is believed that copolymers with controlled amphiphilic properties are able to insert the hydrophobic chain into a mechanically disrupted membrane which has a lower density of lipid packing, but are repelled after the membrane is sealed. However, in vivo decomposition of the self-assembled micelles during systemic circulation permits effective delivery of amphiphilic unimers to the injury site.

Polymer micelles are designed to encapsulate hydrophobic anti-inflammatory drugs that effectively suppress the intracellular injury induced by Ca²⁺ influx. After systemic administration, micelles reduce their stability during blood circulation, as shown by Applicant's FRET studies, especially when the loaded drug is released (Chen et al. (2008) Langmuir 24:5213-5217; Chen et al. (2008) Proc Natl Acad Sci USA 105:6596-6601, both incorporated herein by reference). Both unimers and anti-inflammatory drugs are delivered to the injury site through the compromised blood-spinal cord barrier.

Furthermore, the polymer micelle-based membrane repair method is challenged by the narrow therapeutic time window in clinical applications. For instance, the micelles have to be administered before the secondary neuronal injury becomes dominant. Thus, an alternative treatment option for the treatment of neural injuries and diseases is highly desired.

The present disclosure demonstrates that problems for the treatment of neural injuries and diseases can be overcome using therapeutic compositions with dual actions of 1) repair of damaged membrane and 2) suppression of intracellular inflammation action. The described compositions may act synergistically to rescue more neural cells from injury induced cell death and further extend the therapeutic window for intervention as compared to treatments having only a single action.

The present disclosure describes hydrophobically modified nanoparticles and polymeric nanostructures that can be utilized to for the treatment of neural injury or neural disease in an affected patient, along with methods of forming and using the nanoparticles and nano structures.

The hydrophobically modified nanoparticles and polymeric nanostructures according to the present disclosure provide several advantages compared to alternatives known in the art. First, the nanoparticles and nanostructures of the present disclosure are designed as “dual action” compositions to treat neural injury and neural disease via repair of damaged membrane and suppression of intracellular inflammation.

Second, the nanoparticles and nanostructures of the present disclosure have improved pharmacokinetic parameters compared to alternatives known in the art. For example, the nanoparticles and nanostructures may be associated with a more targeted delivery to the site in need of repair or treatment, and may be associated with a reduction in potentially harmful side effects and/or toxicities at other sites of the body.

Third, compared to other PEG embodiments used in the art, the nanoparticles and nanostructures of the present disclosure are hydrophobically modified or include a hydrophobic domain, respectively. The inclusion of a hydrophobic moiety enhances the effectiveness of the compositions due to a slower rate of clearance from the body after systemic administration.

Fourth, in the embodiments in which the nanoparticles and nanostructures of the present disclosure include an anti-inflammatory agent, the resultant composition may be administered to a patient as a single agent without the need for separate administrations of the nanoparticles/nanostructures and the anti-inflammatory agent.

Finally, the nanoparticles and nanostructures of the present disclosure may have improved loading efficiency of an anti-inflammatory agent in order to facilitate a more potent and targeted delivery of the anti-inflammatory agent to the site in need of repair or treatment.

The following numbered embodiments are contemplated and are non-limiting:

1. A composition comprising a hydrophobically modified nanoparticle comprising a polysaccharide and a pharmacophore, wherein the polysaccharide is covalently bound to the pharmacophore.

2. The composition of clause 1 or clause 2 wherein the polysaccharide is covalently bound to the pharmacophore via an amide bond.

3. The composition of clause 1 or clause 2 wherein the polysaccharide is chitosan.

4. The composition of clause 1 or clause 2 wherein the polysaccharide is a chitosan derivative.

5. The composition of clause 1 or clause 2 wherein the polysaccharide is glycol chitosan.

6. The composition of any one of clauses 1 to 5 wherein the pharmacophore is a fatty acid.

7. The composition of any one of clauses 1 to 5 wherein the pharmacophore is cholanic acid.

8. The composition of any one of clauses 1 to 5 wherein the pharmacophore is ferulic acid.

9. The composition of any one of clauses 1 to 5 wherein the pharmacophore is a ferulic acid derivative.

10. The composition of clause 1 wherein the polysaccharide is glycol chitosan and the pharmacophore is ferulic acid.

11. The composition of clause 10 wherein the nanoparticle has a degree of substitution of ferulic acid per glycol chitosan (ferulic acid:glycol chitosan chain) selected from the group consisting of 5:1, 11:1, and 21:1.

12. The composition of clause 10 wherein the nanoparticle has a degree of substitution of ferulic acid per glycol chitosan (ferulic acid:glycol chitosan chain) of 11:1.

13. The composition of any one of clauses 1 to 12 further comprising a therapeutically effective amount of an anti-inflammatory agent.

14. The composition of clause 13 wherein the anti-inflammatory agent is a corticosteroid.

15. The composition of clause 14 wherein the corticosteroid is selected from the group consisting of betamethasone, dexamethasone, flumethasone, methylprednisolone, paramethasone, prednisolone, prednisone, triamcinolone, hydrocortisone, and cortisone.

16. The composition of clause 14 wherein the corticosteroid is methylprednisolone.

17. The composition of clause 13 wherein the anti-inflammatory agent is curcumin.

18. The composition of clause 13 wherein the pharmacophore is cholanic acid and the anti-inflammatory agent is methylprednisolone.

19. The composition of clause 13 wherein the pharmacophore is ferulic acid and the anti-inflammatory agent is curcumin.

20. The composition of any one of clauses 1 to 19 wherein the average diameter of the nanoparticle is about 100 to about 500 nanometers (nm).

21. The composition of any one of clauses 1 to 19 wherein the average diameter of the nanoparticle is about 200 to about 400 nanometers (nm).

22. The composition of any one of clauses 1 to 19 wherein the average diameter of the nanoparticle is about 300 nanometers (nm).

23. The composition of any one of clauses 1 to 19 wherein the average diameter of the nanoparticle is about 320 nanometers (nm).

24. The composition of any one of clauses 1 to 19 wherein the average diameter of the nanoparticle is about 350 nanometers (nm).

25. The composition of any one of clauses 1 to 24 for use in the treatment of a neuronal injury.

26. The composition of any one of clauses 1 to 24 for use in the treatment of a spinal cord injury.

27. The composition of any one of clauses 1 to 24 for use in the treatment of a traumatic brain injury.

28. The composition of any one of clauses 1 to 24 for use in the contact an injured nerve.

29. The composition of any one of clauses 1 to 24 for use in the repair of an injured nerve.

30. The composition of any one of clauses 1 to 24 for use as a neuroprotective agent.

31. The composition of any one of clauses 1 to 30 wherein the composition is associated with an improvement in a pharmacokinetic parameter in a patient.

32. The composition of any one of clauses 1 to 30 wherein the composition is associated with a reduction in organ toxicity in a patient.

33. The composition of any one of clauses 1 to 30 wherein the composition is associated with a reduction in kidney damage in a patient.

34. A composition comprising a polymeric nanostructure comprising a hydrophobic core, a hydrophilic shell, and a therapeutically effective amount of an anti-inflammatory agent.

35. The composition of clause 34 wherein the nanostructure is a micelle.

36. The composition of clause 34 or clause 35 wherein the hydrophobic core harbors the anti-inflammatory agent.

37. The composition of any one of clauses 34 to 36 wherein the hydrophilic shell comprises a monomethoxy poly(ethylene glycol) (mPEG).

38. The composition of any one of clauses 34 to 37 wherein the hydrophobic core comprises a polyester.

39. The composition of clause 38 wherein the polyester is selected from the group consisting of a poly ε-caprolactone (PCL), a poly lactic-glycolytic acid (PLGA), a poly lactic acid (PLA), and a poly(D,L-lactic acid) (PDLLA).

40. The composition of clause 38 wherein the polyester is PLGA.

41. The composition of any one of clauses 34 to 40 wherein the anti-inflammatory agent is a corticosteroid.

42. The composition of clause 41 wherein the corticosteroid is selected from the group consisting of betamethasone, dexamethasone, flumethasone, methylprednisolone, paramethasone, prednisolone, prednisone, triamcinolone, hydrocortisone, and cortisone.

43. The composition of clause 41 wherein the corticosteroid is methylprednisolone.

44. The composition of any one of clauses 34 to 40 wherein the anti-inflammatory agent is curcumin.

45. The composition of any one of clauses 34 to 44 wherein the average diameter of the nanostructure is about 10 to about 200 nanometers (nm).

46. The composition of any one of clauses 34 to 44 wherein the average diameter of the nanostructure is about 50 to about 150 nanometers (nm).

47. The composition of any one of clauses 34 to 44 wherein the average diameter of the nanostructure is about 60 nanometers (nm).

48. The composition of any one of clauses 34 to 44 wherein the average diameter of the nanostructure is about 120 nanometers (nm).

49. The composition of any one of clauses 34 to 48 for use in the treatment of a neuronal injury.

50. The composition of any one of clauses 34 to 48 for use in the treatment of a spinal cord injury.

51. The composition of any one of clauses 34 to 48 for use in the treatment of a traumatic brain injury.

52. The composition of any one of clauses 34 to 48 for use in the contact an injured nerve.

53. The composition of any one of clauses 34 to 48 for use in the repair of an injured nerve.

54. The composition of any one of clauses 34 to 48 for use as a neuroprotective agent.

55. The composition of any one of clauses 34 to 54 wherein the composition is associated with an improvement in a pharmacokinetic parameter in a patient.

56. The composition of any one of clauses 34 to 54 wherein the composition is associated with a reduction in organ toxicity in a patient.

57. The composition of any one of clauses 34 to 54 wherein the composition is associated with a reduction in kidney damage in a patient.

58. A composition comprising a polysaccharide nanoparticle comprising a polysaccharide, wherein the polysaccharide has a high molecular weight.

59. The composition of clause 58 wherein the polysaccharide is chitosan.

60. The composition of clause 58 wherein the polysaccharide is a chitosan derivative.

61. The composition of clause 58 wherein the polysaccharide is glycol chitosan.

62. The composition of any one of clauses 58 to 61 wherein the molecular weight of the polysaccharide is between about 50 kDa and about 250 kDa.

63. The composition of any one of clauses 58 to 61 wherein the molecular weight of the polysaccharide is about 100 kDa.

64. The composition of any one of clauses 58 to 61 wherein the molecular weight of the polysaccharide is about 200 kDa.

65. The composition of any one of clauses 58 to 64 further comprising a therapeutically effective amount of an anti-inflammatory agent.

66. The composition of clause 65 wherein the anti-inflammatory agent is a corticosteroid.

67. The composition of clause 66 wherein the corticosteroid is selected from the group consisting of betamethasone, dexamethasone, flumethasone, methylprednisolone, paramethasone, prednisolone, prednisone, triamcinolone, hydrocortisone, and cortisone.

68. The composition of clause 66 wherein the corticosteroid is methylprednisolone.

69. The composition of clause 65 wherein the anti-inflammatory agent is curcumin.

70. The composition of any one of clauses 58 to 69 wherein the average diameter of the nanoparticle is about 100 to about 500 nanometers (nm).

71. The composition of any one of clauses 58 to 70 for use in the treatment of a neuronal injury.

72. The composition of any one of clauses 58 to 70 for use in the treatment of a spinal cord injury.

73. The composition of any one of clauses 58 to 70 for use in the treatment of a traumatic brain injury.

74. The composition of any one of clauses 58 to 70 for use in the contact an injured nerve.

75. The composition of any one of clauses 58 to 70 for use in the repair of an injured nerve.

76. The composition of any one of clauses 58 to 70 for use as a neuroprotective agent.

77. The composition of any one of clauses 58 to 76 wherein the composition is associated with an improvement in a pharmacokinetic parameter in a patient.

78. The composition of any one of clauses 58 to 76 wherein the composition is associated with a reduction in organ toxicity in a patient.

79. The composition of any one of clauses 58 to 76 wherein the composition is associated with a reduction in kidney damage in a patient.

80. A method of treating a patient having a neuronal injury, the method comprising the step of administering to the patient a therapeutically effective amount of the hydrophobically modified nanoparticle of any one of clauses 1 to 33.

81. A method of treating a patient having a neuronal injury, the method comprising the step of administering to the patient a therapeutically effective amount of the polymeric nanostructure of any one of clauses 34 to 57.

82. A method of treating a patient having a neuronal injury, the method comprising the step of administering to the patient a therapeutically effective amount of the polysaccharide nanoparticle of any one of clauses 58 to 79.

83. The method of any one of clauses 80 to 82 wherein the neuronal injury is a spinal cord injury.

84. The method of any one of clauses 80 to 82 wherein the neuronal injury is a traumatic brain injury.

85. The method of any one of clauses 80 to 82 wherein the method is used to contact an injured nerve.

86. The method of any one of clauses 80 to 82 wherein the method is used to repair an injured nerve.

87. The method of any one of clauses 80 to 86 wherein the administration is performed within 48 hours of occurrence of the neuronal injury.

88. The method of any one of clauses 80 to 86 wherein the administration is performed within 24 hours of occurrence of the neuronal injury.

89. The method of any one of clauses 80 to 86 wherein the administration is performed between about 1 hour to about 12 hours of occurrence of the neuronal injury.

90. The method of any one of clauses 80 to 86 wherein the administration is performed within 12 hours of occurrence of the neuronal injury.

91. The method of any one of clauses 80 to 86 wherein the administration is performed within 8 hours of occurrence of the neuronal injury.

92. The method of any one of clauses 80 to 86 wherein the administration is performed within 4 hours of occurrence of the neuronal injury.

93. The method of any one of clauses 80 to 86 wherein the administration is performed within 2 hours of occurrence of the neuronal injury.

94. The method of any one of clauses 80 to 93 wherein the method is associated with an improvement in a pharmacokinetic parameter in the patient.

95. The method of any one of clauses 80 to 93 wherein the method is associated with a reduction in organ toxicity in the patient.

96. The method of any one of clauses 80 to 93 wherein the method is associated with a reduction in kidney damage in the patient.

97. The method of any one of clauses 80 to 93 wherein the method reduces a symptom associated with kidney damage.

98. The method of any one of clauses 80 to 93 wherein the administration is an injection.

99. The method of clause 98 wherein the injection is selected from the group consisting of intraarticular, intravenous, intramuscular, intradermal, intraperitoneal, and subcutaneous injections.

100. The method of clause 99 wherein the injection is an intravenous injection.

101. The method of any one of clauses 80 to 100 wherein the administration is performed as a single dose administration.

102. The method of any one of clauses 80 to 100 wherein the administration is performed as a multiple dose administration.

103. A method of treating a patient having a neuronal disease, the method comprising the step of administering to the patient a therapeutically effective amount of the hydrophobically modified nanoparticle of any one of clauses 1 to 33.

104. A method of treating a patient having a neuronal disease, the method comprising the step of administering to the patient a therapeutically effective amount of the polymeric nanostructure of any one of clauses 34 to 57.

105. A method of treating a patient having a neuronal disease, the method comprising the step of administering to the patient a therapeutically effective amount of the polysaccharide nanoparticle of any one of clauses 58 to 79.

106. The method of any one of clauses 103 to 105 wherein the neuronal disease is an acute neuronal disease.

107. The method of any one of clauses 103 to 105 wherein the neuronal disease is a chronic neuronal disease.

108. The method of any one of clauses 103 to 107 wherein the administration is an injection.

109. The method of clause 108 wherein the injection is selected from the group consisting of intraarticular, intravenous, intramuscular, intradermal, intraperitoneal, and subcutaneous injections.

110. The method of clause 109 wherein the injection is an intravenous injection.

111. The method of any one of clauses 103 to 109 wherein the administration is performed as a single dose administration.

112. The method of any one of clauses 103 to 109 wherein the administration is performed as a multiple dose administration.

113. A pharmaceutical formulation comprising the hydrophobically modified nanoparticle of any one of clauses 1 to 33.

114. A pharmaceutical formulation comprising the polymeric nanostructure of any one of clauses 34 to 57.

115. A pharmaceutical formulation comprising the polysaccharide nanoparticle of any one of clauses 58 to 79.

116. The pharmaceutical formulation of any one of clauses 113 to 115 further comprising a pharmaceutically acceptable carrier.

117. The pharmaceutical formulation of any one of clauses 113 to 116 optionally including one or more other therapeutic ingredients.

118. The pharmaceutical formulation of any one of clauses 113 to 117 wherein the formulation is a single unit dose.

119. A lyophilisate or powder of the pharmaceutical formulation of any one of clauses 113 to 118.

120. An aqueous solution produced by dissolving the lyophilisate or powder of clause 119 in water.

### BRIEF DESCRIPTION OF THE DRAWINGS

FIG. 1 shows recovery of locomotor function in SCI rats, measured by Basso Beattie Bresnahan (BBB) score, after intravenous injection of 1 ml of 5 mg/ml curcumin-loaded hydrophobically modified glycol chitosan (HGC) nanoparticles. The loading efficiency is 10%, corresponding to 500 μg/mlcurcumin in the nanoparticle solution. The injection through the jugular vein was performed at 2 hours after a contusion injury of spinal cord.

FIG. 2 shows pharmacokinetics demonstrating the half-life of HGC nanoparticles in blood.

FIG. 3 shows an exemplary synthesis of curcumin-loaded HGC nanoparticles. (a) Ferulic acid is a product of curcumin hydrolysis. (b) Synthetic scheme for conjugation between GC and FA. (c) Schematic illustration of curcumin-loaded HGC nanoparticles. (d) Solubility test of curcumin in PBS without (left) or with HGC (right).

FIG. 4 shows the photoacoustic membrane poration model. (a) Setup. (b) Membrane integrity test with calcein AM (green) and propidium iodide (red). After irradiation, the cells in the area within the laser spot were damaged, labeled with propidium iodide, while the cells out of the irradiation area were still healthy, labeled with calcein. (c) A zoomed-in image showing membrane blebbing of a cell after irradiation. Cell nucleus was labeled by propidium iodide. Bar=10 μm.

FIG. 5 shows a double sucrose gap recording chamber for the recordation of CAPs.

FIG. 6 shows a flowchart of in vivo studies for spinal injury and repair.

FIG. 7 shows precipitation of loaded curcumin in FA-GC nanoparticles with degree of substitution (DS)=21 (see right panels). In contrast, the FA-GC with DS=11 was capable of stably encapsulating curcumin (see left panels).

FIG. 8 shows (a) Glycol chitosan chemically conjugated with ferulic acid (FA), a product of curcumin hydrolysis; (b) the average diameter of the cucumin-loaded GC-FA nanoparticles by transmission electron microscopy (TEM); (c) the average diameter of the cucumin-loaded GC-FA nanoparticles by dynamic light scattering (DLS); (d) co-localization of fluorescence signals from curcumin (left, green) and Cy5.5-labeled FA-GC (right, red); (e) precipitation over one month for the curcumin present in FA-GC.

FIG. 9 shows detection of curcumin and warfarin by their ionized fragments (m/z=149 for curcumin, m/z=161 for warfarin) in the mass spectra.

FIG. 10 shows (a) concentration of curcumin using a calibration curve derived from the ratio between mass intensities of curcumin and warfarin; (b) the concentration of curcumin in the injured cord compared to the normal cord; (c) blood retention time determined by the one-compartment model; (d) the signal observed at the lesion site of the spinal cord.

FIG. 11 shows curcumin in FA-GC nanoparticles is mostly eliminated through the kidney.

FIG. 12 shows the half-life of non-modified GC.

FIG. 13 shows the fluorescence intensity at the injured spinal cord compared to other organs.

FIG. 14 shows (a) the fluorescence signal inside the gray matter that is highly vulnerable to a contusive injury (see the formation of cavities); (b) the myelin sheath in posterior white matter demonstrates irregular morphology; (c) the myelin sheath near central canal demonstrates irregular morphology; (d) high magnification SRS image of the gray matter demonstrates clots of red blood cells; (e) the myelin sheath in the anterior white matter is highly convoluted exhibited.

FIG. 15 shows curcumin enters cells and GC-FA targets the cell membrane after a 4 hour incubation with GC-FA nanoparticles.

FIG. 16 shows (a) confocal imaging of the cell membrane attachment of GC-FA and cellular internalization of curcumin; (b) treatment with 0.2 mg/ml GC-FA/curcumin significantly reduced the number of PI stained cell; (c) GC-FA/curcumin treatment increased the survival rate from 20% to 95% and GC-FA alone helped rescue the cells by 55%; (d) all three treatments significantly protected PC12 cells in the glutamate damage model.

FIG. 17 shows recovery of locomotor function in treated rats.

FIG. 18 shows reduction of levels of magnesium and BUN after FA-GC treatment.

FIG. 19 shows identification of astrocyte and macrophage/activated microglia via GFAP and ED-1.

FIG. 20 shows (a) the cavity area indicated by astrocyte boundary in saline treated animals; (b) the activated astrocytes and activated microglia the fluorescence of GFAP in the epicenter of the lesion in saline treated animals; (c) the activated astrocytes and activated microglia the fluorescence of ED-1 in the epicenter of the lesion in saline treated animals; (d) the cavity area indicated by astrocyte boundary in nanoparticle treated animals; (e) the activated astrocytes and activated microglia the fluorescence of GFAP in the epicenter of the lesion in nanoparticle treated animals; (f) the activated astrocytes and activated microglia the fluorescence of ED-1 in the epicenter of the lesion in nanoparticle treated animals; (m) the GFAP fluorescence significantly reduced in FA-GC/curcumin treated group compare to saline treated group (187.38±46.37 vs. 339.37±49.47); (n) the ED-1 fluorescence significantly reduced in FA-GC/curcumin treated group compare to saline treated group (103.20±39.67 vs. 242.35±55.38); (O) the cavity area significantly decreased in the nanoparticle treated group (1.67±0.5 mm²) compared to the saline treated group (5.19±0.92 mm²).

FIG. 21 shows safety analysis of curcumin-loaded FA-GC nanoparticles compared to saline treatment.

### DETAILED DESCRIPTION OF THE ILLUSTRATIVE EMBODIMENTS

As used herein, a “hydrophobically modified nanoparticle” means a nanoparticle that has been modified with a hydrophobic moiety. As used herein, a “polymeric nanostructure” means a nanostructure comprised of one or more polymers. A nanoparticle or a nanostructure is understood by those of skill in the art to refer to a particle having at least one dimension of submicron size.

Various embodiments of the invention are described herein as follows. In one embodiment described herein, a hydrophobically modified nanoparticle is provided. The hydrophobically modified nanoparticle comprises a polysaccharide and a pharmacophore, wherein the polysaccharide is covalently bound to the pharmacophore.

In another embodiment, a polymeric nanostructure is provided. The polymeric nanostructure comprises a hydrophobic core, a hydrophilic shell, and a therapeutically effective amount of an anti-inflammatory agent.

In another embodiment, a polysaccharide nanoparticle is provided. The polysaccharide nanoparticle comprises a polysaccharide, wherein the polysaccharide has a high molecular weight.

In other embodiments, methods of treatment for a neural injury in a patient are provided. In one illustrative embodiment, the method comprises the step of administering to the patient a therapeutically effective amount of the hydrophobically modified nanoparticle. In another illustrative embodiment, the method comprises the step of administering to the patient a therapeutically effective amount of the polymeric nanostructure. In a further illustrative embodiment, the method comprises the step of administering to the patient a therapeutically effective amount of the polysaccharide nanoparticle.

In other embodiments, methods of treatment for a neural disease in a patient are provided. In one illustrative embodiment, the method comprises the step of administering to the patient a therapeutically effective amount of the hydrophobically modified nanoparticle. In another illustrative embodiment, the method comprises the step of administering to the patient a therapeutically effective amount of the polymeric nanostructure. In a further illustrative embodiment, the method comprises the step of administering to the patient a therapeutically effective amount of the polysaccharide nanoparticle.

In yet other embodiments, pharmaceutical formulations are provided. In one illustrative embodiment, the pharmaceutical formulation comprises the hydrophobically modified nanoparticle. In another illustrative embodiment, the pharmaceutical formulation comprises the polymeric nanostructure. In yet another illustrative embodiment, the pharmaceutical formulation comprises the polysaccharide nanoparticle.

In various embodiments described herein, the polysaccharide component of the hydrophobically modified nanoparticle described herein can be covalently bound to the pharmacophore. In one embodiment, the polysaccharide is bound to the pharmacophore via an amide bond.

In some embodiments described herein, the polysaccharide component of the hydrophobically modified nanoparticle described herein is chitosan. In other embodiments described herein, the polysaccharide component of the hydrophobically modified nanoparticle described herein is a chitosan derivative. As used herein, the term “chitosan derivative” refers to a modification of the natural polysaccharide chitosan. In one embodiment described herein, the polysaccharide component of the hydrophobically modified nanoparticle described herein is glycol chitosan.

In other embodiments described herein, the polysaccharide component of the hydrophobically modified nanoparticle described herein is a fatty acid. As used herein, the term “fatty acid” means a carboxylic acid with a long aliphatic tail, and can be either saturated or unsaturated. Examples of fatty acids are well known in the art, for example those derived from triglycerides or phospholipids.

In various embodiments described herein, the pharmacophore component of the hydrophobically modified nanoparticle described herein is cholanic acid. In some embodiments described herein, the pharmacophore component of the hydrophobically modified nanoparticle described herein is a cholanic acid derivative. In other embodiments described herein, the pharmacophore component of the hydrophobically modified nanoparticle described herein is ferulic acid. In some embodiments described herein, the pharmacophore component of the hydrophobically modified nanoparticle described herein is a ferulic acid derivative.

In some embodiments described herein, the polysaccharide component of the hydrophobically modified nanoparticle is glycol chitosan and the pharmacophore component of the hydrophobically modified nanoparticle is ferulic acid. In other embodiments, the nanoparticle has a measured degree of substitution understood by those of skill in the art to refer to the number of ferulic acid per chitosan chain. In some embodiments, the nanoparticle has a degree of substitution of ferulic acid per glycol chitosan (ferulic acid:glycol chitosan chain) selected from the group consisting of 5:1, 11:1, and 21:1. In one embodiments, the nanoparticle has a degree of substitution of ferulic acid per glycol chitosan (ferulic acid:glycol chitosan chain) of 11:1.

In other illustrative embodiments described herein, the hydrophobically modified nanoparticle further comprises a therapeutically effective amount of an anti-inflammatory agent. As used herein, the term “therapeutically effective amount” refers to an amount which gives the desired benefit to an animal and includes both treatment and prophylactic administration. The amount will vary from one animal to another and will depend upon a number of factors, including the overall physical condition of the animal and the underlying cause of the condition to be treated. As used herein, the term “anti-inflammatory agent” refers to any compound that reduces inflammation in a patient and/or reduces the pain or swelling associated with inflammation.

In some embodiments, the anti-inflammatory agent component of the hydrophobically modified nanoparticle is a corticosteroid. In other embodiments, the corticosteroid is selected from the group consisting of betamethasone, dexamethasone, flumethasone, methylprednisolone, paramethasone, prednisolone, prednisone, triamcinolone, hydrocortisone, and cortisone. In one embodiment, the corticosteroid is methylprednisolone. In some embodiments, the anti-inflammatory agent component of the hydrophobically modified nanoparticle is curcumin.

The hydrophobicity of the polysaccharide component of the hydrophobically modified nanoparticle may be specifically modified to optimize the loading efficiency and intracellular delivery of anti-inflammatory agent as well the insertion of hydrophobic unimers to damaged membranes. Optimization of the loading efficiency can result in more efficient delivery of the anti-inflammatory agent to the site of need within the body. Furthermore, optimization of the loading efficiency can result in a targeted delivery of the anti-inflammatory agent to the site of need within the body and may avoid harmful side effects or undesired toxicities to other sites within the body.

In various illustrative embodiments described herein, the pharmacophore component of the hydrophobically modified nanoparticle is cholanic acid and the anti-inflammatory agent component of the hydrophobically modified nanoparticle is methylprednisolone. In other illustrative embodiments described herein, the pharmacophore component of the hydrophobically modified nanoparticle is ferulic acid and the anti-inflammatory agent component of the hydrophobically modified nanoparticle is curcumin.

In one illustrative aspect, the pharmacophore is attached to a portion of amine groups of the polysaccharide. In one embodiment, the ferulic acid is bound to a portion of amine groups of glycol chitosan. In one embodiment, the ferulic acid is bound to about 1% to about 30%, about 1% to about 20%, about 5% to about 30%, about 5% to about 20%, about 5% to about 15%, or about 8% to about 15%, about 8% to about 12% of the glycol chitosan amines.

In various embodiments described herein, the hydrophobically modified nanoparticles may have an average diameter in solution of about 10 nm to about 950 nm, about 10 nm to about 700 nm, about 100 nm to about 950 nm, about 100 nm to about 500 nm, about 100 nm to about 400 nm, about 200 nm to about 400 nm, about 250 nm to about 350 nm, or about 300 nm to about 400 nm. These various nanoparticles size ranges are also contemplated where the term “about” is not included. In one embodiment, the hydrophobically modified nanoparticles may have an average diameter of about 200 nanometers. In one embodiment, the hydrophobically modified nanoparticles may have an average diameter of about 250 nanometers. In one embodiment, the hydrophobically modified nanoparticles may have an average diameter of about 300 nanometers. In one embodiment, the hydrophobically modified nanoparticles may have an average diameter of about 320 nanometers. In one embodiment, the hydrophobically modified nanoparticles may have an average diameter of about 350 nanometers. In one embodiment, the hydrophobically modified nanoparticles may have an average diameter of about 400 nanometers.

In various embodiments described herein, the hydrophobically modified nanoparticles may be for use in the treatment of a neural injury. In other embodiments described herein, the hydrophobically modified nanoparticles may be for use in the treatment of a spinal cord injury. In yet other embodiments described herein, the hydrophobically modified nanoparticles may be for use in the treatment of a traumatic brain injury. In other embodiments described herein, the hydrophobically modified nanoparticles may be for use to contact an injured nerve. In yet other embodiments described herein, the hydrophobically modified nanoparticles may be for use to repair an injured nerve. In other embodiments described herein, the hydrophobically modified nanoparticles may be for use as a neuroprotective agent.

In some embodiments described herein, the hydrophobically modified nanoparticles may be associated with an improvement in a pharmacokinetic parameter in a patient. In one embodiment, the pharmacokinetic parameter that is improved is the absorption of the polymeric nanostructure in a patient. In another embodiment, the pharmacokinetic parameter that is improved is the distribution of the polymeric nanostructure in a patient. In yet another embodiment, the pharmacokinetic parameter that is improved is the delivery of the polymeric nanostructure in a patient. In one embodiment, the pharmacokinetic parameter that is improved is the elimination of the polymeric nanostructure in a patient. In another embodiment, the pharmacokinetic parameter that is improved is the reduction in organ toxicity in a patient. In yet another embodiment, the pharmacokinetic parameter that is improved is the reduction in kidney toxicity in a patient. In another embodiment, the pharmacokinetic parameter that is improved is the reduction in kidney damage in a patient.

In another embodiment, a polymeric nanostructure is provided. The polymeric nanostructure comprises a hydrophobic core, a hydrophilic shell, and a therapeutically effective amount of an anti-inflammatory agent.

In various embodiments described herein, the polymeric nanostructure described herein is a micelle. As used herein, the term “micelle” means an aggregate of amphipathic molecules in water, wherein the nonpolar portions are in the interior and the polar portions are at the exterior surface.

In some illustrative embodiments described herein, the polymeric nanostructure described herein harbors the anti-inflammatory agent. As used herein, the term “harbor” includes linked, attached, bound, conjugated, and the like, including partially to completely encapsulated. In one embodiment, the anti-inflammatory agent is harbored in the hydrophobic domain of the polymeric nanostructure.

In some embodiments, the hydrophilic shell component of the polymeric nanostructure comprises a monomethoxy poly(ethylene glycol) (mPEG). In one illustrative aspect, the molecular weight of the mPEG is about 1000 Da to 5000 Da, about 1500 Da to about 4000 Da, about 2000 Da to about 5000 Da, about 2000 Da to about 3000 Da, or about 1500 Da to about 2500 Da. These mPEG size ranges are also contemplated where the term “about” is not included.

In some embodiments, the hydrophobic core component of the polymeric nanostructure comprises a polyester. In other embodiments, the polyester is selected from the group consisting of a poly ε-caprolactone (PCL), a poly lactic-glycolytic acid (PLGA), a poly lactic acid (PLA), and a poly(D,L-lactic acid) (PDLLA). In one embodiment, the polyester is PLGA. In one illustrative aspect, the PCL, PLGA, PLA, or PDLLA has a molecular weight of about 2000 Da to about 20,000 Da, about 4000 Da to about 20,000 Da, about 2000 Da, to about 16,000 Da, about 4000 Da to about 16,000 Da, about 8000 Da to about 16,000 Da, or about 4000 Da to about 8000 Da. These size ranges are also contemplated where the term “about” is not included. Any combination of above molecular weights of mPEG and molecular weights PCL, PLGA, PLA, or PDLLA is contemplated.

In some embodiments, the anti-inflammatory agent component of the polymeric nanostructure is a corticosteroid. In other embodiments, the corticosteroid is selected from the group consisting of betamethasone, dexamethasone, flumethasone, methylprednisolone, paramethasone, prednisolone, prednisone, triamcinolone, hydrocortisone, and cortisone. In one embodiment, the corticosteroid is methylprednisolone. In some embodiments, the anti-inflammatory agent component of the polymeric nanostructure is curcumin.

In various embodiments described herein, the polymeric nanostructures may have an average diameter in solution of about 10 nm to about 950 nm, about 10 nm to about 700 nm, about 10 nm to about 200 nm, about 50 nm to about 150 nm, about 100 nm to about 950 nm, about 100 nm to about 500 nm, about 100 nm to about 400 nm, about 200 nm to about 400 nm, about 250 nm to about 350 nm, or about 300 nm to about 400 nm. These various nanostructures size ranges are also contemplated where the term “about” is not included. In one embodiment, the polymeric nanostructures may have an average diameter of about 200 nanometers. In one embodiment, the polymeric nanostructures may have an average diameter of about 150 nanometers. In one embodiment, the polymeric nanostructures may have an average diameter of about 120 nanometers. In one embodiment, the polymeric nanostructures may have an average diameter of about 100 nanometers. In one embodiment, the polymeric nanostructures may have an average diameter of about 60 nanometers. In one embodiment, the polymeric nanostructures may have an average diameter of about 50 nanometers.

In various embodiments described herein, the polymeric nanostructures may be for use in the treatment of a neural injury. In other embodiments described herein, the polymeric nanostructures may be for use in the treatment of a spinal cord injury. In yet other embodiments described herein, the polymeric nano structures may be for use in the treatment of a traumatic brain injury. In other embodiments described herein, the polymeric nanostructures may be for use to contact an injured nerve. In yet other embodiments described herein, the polymeric nanostructures may be for use to repair an injured nerve. In other embodiments described herein, the polymeric nanostructures may be for use as a neuroprotective agent.

In some embodiments described herein, the polymeric nanostructures may be associated with an improvement in a pharmacokinetic parameter in a patient. In one embodiment, the pharmacokinetic parameter that is improved is the absorption of the polymeric nanostructure in a patient. In another embodiment, the pharmacokinetic parameter that is improved is the distribution of the polymeric nanostructure in a patient. In yet another embodiment, the pharmacokinetic parameter that is improved is the delivery of the polymeric nanostructure in a patient. In one embodiment, the pharmacokinetic parameter that is improved is the elimination of the polymeric nano structure in a patient. In another embodiment, the pharmacokinetic parameter that is improved is the reduction in organ toxicity in a patient. In yet another embodiment, the pharmacokinetic parameter that is improved is the reduction in kidney toxicity in a patient. In another embodiment, the pharmacokinetic parameter that is improved is the reduction in kidney damage in a patient.

In another embodiment, a polysaccharide nanoparticle is provided. The polysaccharide nanoparticle comprises a polysaccharide, wherein the polysaccharide has a high molecular weight.

In various embodiments described herein, the polysaccharide has a high molecular weight. For example, the molecular weight of the polysaccharide may be between about 50 kDa and about 250 kDa. In some embodiments, the molecular weight of the polysaccharide is about 50 kDa. In other embodiments, the molecular weight of the polysaccharide is about 75 kDa. In yet other embodiments, the molecular weight of the polysaccharide is about 100 kDa. In some embodiments, the molecular weight of the polysaccharide is about 125 kDa. In other embodiments, the molecular weight of the polysaccharide is about 150 kDa. In yet other embodiments, the molecular weight of the polysaccharide is about 200 kDa. In some embodiments, the molecular weight of the polysaccharide is about 250 kDa.

In some embodiments described herein, the polysaccharide component of the polysaccharide nanoparticle described herein is chitosan. In other embodiments described herein, the polysaccharide component of the polysaccharide nanoparticle described herein is a chitosan derivative. In one embodiment described herein, the polysaccharide component of the polysaccharide nanoparticle described herein is glycol chitosan. In other embodiments described herein, the polysaccharide component of the polysaccharide nanoparticle described herein is a fatty acid.

In other illustrative embodiments described herein, the polysaccharide nanoparticle further comprises a therapeutically effective amount of an anti-inflammatory agent. In some embodiments, the anti-inflammatory agent component of the polysaccharide nanoparticle is a corticosteroid. In other embodiments, the corticosteroid is selected from the group consisting of betamethasone, dexamethasone, flumethasone, methylprednisolone, paramethasone, prednisolone, prednisone, triamcinolone, hydrocortisone, and cortisone. In one embodiment, the corticosteroid is methylprednisolone. In some embodiments, the anti-inflammatory agent component of the polysaccharide nanoparticle is curcumin.

In various embodiments described herein, the polysaccharide nanoparticles may have an average diameter in solution of about 10 nm to about 950 nm, about 10 nm to about 700 nm, about 100 nm to about 950 nm, about 100 nm to about 500 nm, about 100 nm to about 400 nm, about 200 nm to about 400 nm, about 250 nm to about 350 nm, or about 300 nm to about 400 nm. These various nanoparticles size ranges are also contemplated where the term “about” is not included. In one embodiment, the polysaccharide nanoparticles may have an average diameter of about 200 nanometers. In one embodiment, the polysaccharide nanoparticles may have an average diameter of about 250 nanometers. In one embodiment, the polysaccharide nanoparticles may have an average diameter of about 300 nanometers. In one embodiment, the polysaccharide nanoparticles may have an average diameter of about 320 nanometers. In one embodiment, the polysaccharide nanoparticles may have an average diameter of about 350 nanometers. In one embodiment, the polysaccharide nanoparticles may have an average diameter of about 400 nanometers.

In various embodiments described herein, the polysaccharide nanoparticles may be for use in the treatment of a neural injury. In other embodiments described herein, the polysaccharide nanoparticles may be for use in the treatment of a spinal cord injury. In yet other embodiments described herein, the polysaccharide nanoparticles may be for use in the treatment of a traumatic brain injury. In other embodiments described herein, the polysaccharide nanoparticles may be for use to contact an injured nerve. In yet other embodiments described herein, the polysaccharide nanoparticles may be for use to repair an injured nerve. In other embodiments described herein, the polysaccharide nanoparticles may be for use as a neuroprotective agent.

In some embodiments described herein, the polysaccharide nanoparticles may be associated with an improvement in a pharmacokinetic parameter in a patient. In one embodiment, the pharmacokinetic parameter that is improved is the absorption of the polymeric nanostructure in a patient. In another embodiment, the pharmacokinetic parameter that is improved is the distribution of the polymeric nanostructure in a patient. In yet another embodiment, the pharmacokinetic parameter that is improved is the delivery of the polymeric nanostructure in a patient. In one embodiment, the pharmacokinetic parameter that is improved is the elimination of the polymeric nano structure in a patient. In another embodiment, the pharmacokinetic parameter that is improved is the reduction in organ toxicity in a patient. In yet another embodiment, the pharmacokinetic parameter that is improved is the reduction in kidney toxicity in a patient. In another embodiment, the pharmacokinetic parameter that is improved is the reduction in kidney damage in a patient.

In various embodiments, methods of treatment for a neural injury in a patient are provided. In one illustrative embodiment, the method comprises the step of administering to the patient a therapeutically effective amount of the hydrophobically modified nanoparticle. In another illustrative embodiment, the method comprises the step of administering to the patient a therapeutically effective amount of the polymeric nanostructure. In yet another illustrative embodiment, the method comprises the step of administering to the patient a therapeutically effective amount of the polysaccharide nanoparticle. The previously described embodiments of the hydrophobically modified nanoparticle, the polymeric nanostructure, and the polysaccharide nanoparticle are applicable to the method described herein.

In some embodiments, the neural injury to be treated by the described methods is a spinal cord injury. In other embodiments, the neural injury to be treated by the described methods is a traumatic brain injury. In yet other embodiments, the neural injury to be treated by the described methods is repair of an injured nerve.

In some embodiments, the administration according to the described methods is performed within 48 hours of occurrence of the neural injury. In other embodiments, the administration according to the described methods is performed within 24 hours of occurrence of the neural injury. In yet other embodiments, the administration according to the described methods is performed between about 1 hour to about 12 hours of occurrence of the neural injury. In other embodiments, the administration according to the described methods is performed within 12 hours of occurrence of the neural injury. In other embodiments, the administration according to the described methods is performed within 8 hours of occurrence of the neural injury. In other embodiments, the administration according to the described methods is performed within 6 hours of occurrence of the neural injury. In other embodiments, the administration according to the described methods is performed within 4 hours of occurrence of the neural injury. In other embodiments, the administration according to the described methods is performed within 2 hours of occurrence of the neural injury. In other embodiments, the administration according to the described methods is performed within 1 hour of occurrence of the neural injury.

In some embodiments described herein, the described methods may be associated with an improvement in a pharmacokinetic parameter in a patient. In one embodiment, the pharmacokinetic parameter that is improved is the absorption of the polymeric nanostructure in a patient. In another embodiment, the pharmacokinetic parameter that is improved is the distribution of the polymeric nanostructure in a patient. In yet another embodiment, the pharmacokinetic parameter that is improved is the delivery of the polymeric nanostructure in a patient. In one embodiment, the pharmacokinetic parameter that is improved is the elimination of the polymeric nano structure in a patient. In another embodiment, the pharmacokinetic parameter that is improved is the reduction in organ toxicity in a patient. In yet another embodiment, the pharmacokinetic parameter that is improved is the reduction in kidney toxicity in a patient. In another embodiment, the pharmacokinetic parameter that is improved is the reduction in kidney damage in a patient.

In various embodiments, the administration according to the described methods is an injection. In some embodiments, the injection is selected from the group consisting of intraarticular, intravenous, intramuscular, intradermal, intraperitoneal, and subcutaneous injections. In one embodiment, the injection is an intravenous injection.

In other various embodiments, the administration according to the described methods is performed as a single dose administration. In other embodiments, the administration according to the described methods is performed as a multiple dose administration.

In various embodiments, methods of treatment for a neural disease in a patient are provided. In one illustrative embodiment, the method comprises the step of administering to the patient a therapeutically effective amount of the hydrophobically modified nanoparticle. In another illustrative embodiment, the method comprises the step of administering to the patient a therapeutically effective amount of the polymeric nanostructure. In yet another illustrative embodiment, the method comprises the step of administering to the patient a therapeutically effective amount of the polysaccharide nanoparticle. The previously described embodiments of the hydrophobically modified nanoparticle, the polymeric nanostructure, and the polysaccharide nanoparticle are applicable to the method described herein.

In some embodiments, the neural disease to be treated by the described methods is an acute neural disease. In other embodiments, the neural injury to be treated by the described methods is a chronic neural disease.

In some embodiments described herein, the described methods may be associated with an improvement in a pharmacokinetic parameter in a patient. In one embodiment, the pharmacokinetic parameter that is improved is the absorption of the polymeric nanostructure in a patient. In another embodiment, the pharmacokinetic parameter that is improved is the distribution of the polymeric nanostructure in a patient. In yet another embodiment, the pharmacokinetic parameter that is improved is the delivery of the polymeric nanostructure in a patient. In one embodiment, the pharmacokinetic parameter that is improved is the elimination of the polymeric nano structure in a patient. In another embodiment, the pharmacokinetic parameter that is improved is the reduction in organ toxicity in a patient. In yet another embodiment, the pharmacokinetic parameter that is improved is the reduction in kidney toxicity in a patient. In another embodiment, the pharmacokinetic parameter that is improved is the reduction in kidney damage in a patient.

In various embodiments, the administration according to the described methods is an injection. In some embodiments, the injection is selected from the group consisting of intraarticular, intravenous, intramuscular, intradermal, intraperitoneal, and subcutaneous injections. In one embodiment, the injection is an intravenous injection.

In other various embodiments, the administration according to the described methods is performed as a single dose administration. In other embodiments, the administration according to the described methods is performed as a multiple dose administration.

In various embodiments, pharmaceutical formulations are provided. In one illustrative embodiment, the pharmaceutical formulation comprises the hydrophobically modified nanoparticle. In another illustrative embodiment, the pharmaceutical formulation comprises the polymeric nanostructure. In yet another illustrative embodiment, the pharmaceutical formulation comprises the polysaccharide nanoparticle.

In some embodiments, the pharmaceutical formulations described herein further comprise a pharmaceutically acceptable carrier. In some embodiments, the pharmaceutical formulations described herein further comprise a pharmaceutically acceptable diluent. Diluent or carrier ingredients used in the compositions containing nanoparticles or nanostructures can be selected so that they do not diminish the desired effects of the nanoparticle or nanostructure. Examples of suitable dosage forms include aqueous solutions of the nanoparticles or nanostructures, for example, a solution in isotonic saline, 5% glucose or other well-known pharmaceutically acceptable liquid carriers such as alcohols, glycols, esters and amides.

“Carrier” is used herein to describe any ingredient other than the active component(s) in a formulation. Pharmaceutically acceptable carriers are determined in part by the particular composition being administered, as well as by the particular method used to administer the composition (see, e.g., Remington's Pharmaceutical Sciences, 17th ed. 1985)). The choice of carrier will to a large extent depend on factors such as the particular mode of administration, the effect of the carrier on solubility and stability, and the nature of the dosage form. In one illustrative aspect, the carrier is a liquid carrier.

As used herein, the term “pharmaceutically acceptable” includes “veterinarily acceptable”, and thus includes both human and animal applications independently. For example, a “patient” as referred to herein can be a human patient or a veterinary patient, such as a domesticated animal (e.g., a pet).

In some embodiments, the pharmaceutical formulations described herein optionally include one or more other therapeutic ingredients. As used herein, the term “active ingredient” or “therapeutic ingredient” refers to a therapeutically active compound, as well as any prodrugs thereof and pharmaceutically acceptable salts, hydrates, and solvates of the compound and the prodrugs. Other active ingredients may be combined with the described nanoparticles or nanostructures and may be either administered separately or in the same pharmaceutical formulation. The amount of other active ingredients to be given may be readily determined by one skilled in the art based upon therapy with described nanoparticles or nanostructures.

In some embodiments, the pharmaceutical formulations described herein are a single unit dose. As used herein, the term “unit dose” is a discrete amount of the composition comprising a predetermined amount of the described nanoparticles or nanostructures. The amount of the described nanoparticles or nanostructures is generally equal to the dosage of the described nanoparticles or nanostructures which would be administered to an animal or a convenient fraction of such a dosage such as, for example, one-half or one-third of such a dosage.

Pharmaceutically acceptable salts, and common methodologies for preparing pharmaceutically acceptable salts, are known in the art and are included in the definition of the compositions described herein. See, e.g., P. Stahl, et al., HANDBOOK OF PHARMACEUTICAL SALTS: PROPERTIES, SELECTION AND USE, (VCHA/Wiley-VCH, 2002); S. M. Berge, et al., “Pharmaceutical Salts,” Journal of Pharmaceutical Sciences, Vol. 66, No. 1, January 1977.

The compositions described herein and their salts may be formulated as pharmaceutical compositions for systemic administration. Such pharmaceutical compositions and processes for making the same are known in the art for both humans and non-human mammals. See, e.g., REMINGTON: THE SCIENCE AND PRACTICE OF PHARMACY, (1995) A. Gennaro, et al., eds., 19^{th} ed., Mack Publishing Co. Additional active ingredients may be included in the pharmaceutical formulation comprising a nanoparticle or a nanostructure, or a salt thereof.

In one illustrative embodiment, pharmaceutical formulations for use with a hydrophobically modified nanoparticle for parenteral administration comprise: a) a hydrophobically modified nanoparticle; b) a pharmaceutically acceptable pH buffering agent to provide a pH in the range of about pH 4.5 to about pH 9; c) an ionic strength modifying agent in the concentration range of about 0 to about 300 millimolar; and d) a water soluble viscosity modifying agent in the concentration range of about 0.25% to about 10% total formula weight or any combinations of a), b), c) and d) are provided.

In one illustrative embodiment, pharmaceutical formulations for use with a polymeric nanostructure for parenteral administration comprise: a) a polymeric nanostructure; b) a pharmaceutically acceptable pH buffering agent to provide a pH in the range of about pH 4.5 to about pH 9; c) an ionic strength modifying agent in the concentration range of about 0 to about 300 millimolar; and d) a water soluble viscosity modifying agent in the concentration range of about 0.25% to about 10% total formula weight or any combinations of a), b), c) and d) are provided.

In one illustrative embodiment, pharmaceutical formulations for use with a polysaccharide nanoparticle for parenteral administration comprise: a) a polysaccharide nanoparticle; b) a pharmaceutically acceptable pH buffering agent to provide a pH in the range of about pH 4.5 to about pH 9; c) an ionic strength modifying agent in the concentration range of about 0 to about 300 millimolar; and d) a water soluble viscosity modifying agent in the concentration range of about 0.25% to about 10% total formula weight or any combinations of a), b), c) and d) are provided.

In various illustrative embodiments, the pH buffering agents for use in the compositions and methods herein described are those agents known to the skilled artisan and include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, cacodylate, or MES.

In another illustrative embodiment, the ionic strength modulating agents include those agents known in the art, for example, glycerin, propylene glycol, mannitol, glucose, dextrose, sorbitol, sodium chloride, potassium chloride, and other electrolytes.

Useful viscosity modulating agents include but are not limited to, ionic and non-ionic water soluble polymers; crosslinked acrylic acid polymers such as the “carbomer” family of polymers, e.g., carboxypolyalkylenes that may be obtained commercially under the Carbopol® trademark; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; gums such as tragacanth and xanthan gum; sodium alginate; gelatin, hyaluronic acid and salts thereof, chitosans, gellans or any combination thereof. Typically, non-acidic viscosity enhancing agents, such as a neutral or a basic agent are employed in order to facilitate achieving the desired pH of the formulation.

In one illustrative aspect, parenteral formulations may be suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art.

The aqueous preparations according to the invention can be used to produce lyophilisates by conventional lyophilization or powders. The preparations according to the invention are obtained again by dissolving the lyophilisates in water or other aqueous solutions. The term “lyophilization,” also known as freeze-drying, is a commonly employed technique for presenting proteins which serves to remove water from the protein preparation of interest. Lyophilization is a process by which the material to be dried is first frozen and then the ice or frozen solvent is removed by sublimation in a vacuum environment. An excipient may be included in pre-lyophilized formulations to enhance stability during the freeze-drying process and/or to improve stability of the lyophilized product upon storage. For example, see Pikal, M. Biopharm. 3(9)26-30 (1990) and Arakawa et al. Pharm. Res. 8(3):285-291 (1991).

In one embodiment, the solubility of the nanoparticles or nanostructures used in the preparation of a parenteral formulation may be increased by the use of appropriate formulation techniques, such as the incorporation of solubility-enhancing agents.

In various embodiments, formulations for parenteral administration may be formulated to be for immediate and/or modified release. Modified release formulations include delayed, sustained, pulsed, controlled, targeted and programmed release formulations. Thus, a nanoparticle or a nanostructure may be formulated as a solid, semi-solid, or thixotropic liquid for administration as an implanted depot providing modified release of the active compound.

The formulations can be presented in unit-dose or multi-dose sealed containers, such as ampules and vials. The formulations can also be presented in syringes, such as prefilled syringes.

In various embodiments, the dosages of the nanoparticles or nanostructures can vary significantly depending on the patient condition and the severity of the neural injury or the neural disease. The effective amount to be administered to a patient is based on body surface area, patient weight or mass, and physician assessment of patient condition.

Suitable dosages of the nanoparticles or nanostructures can be determined by standard methods, for example by establishing dose-response curves in laboratory animal models or in humans in clinical trials. Illustratively, suitable dosages of nanoparticles or nanostructures (administered in a single bolus or over time) include from about 1 pg/kg to about 10 μg/kg, from about 1 pg/kg to about 1 μg/kg, from about 100 pg/kg to about 500 ng/kg, from about 1 pg/kg to about 1 ng/kg, from about 1 pg/kg to about 500 pg/kg, from about 100 pg/kg to about 500 ng/kg, from about 100 pg/kg to about 100 ng/kg, from about 1 ng/kg to about 10 mg/kg, from about 1 ng/kg to 1 mg/kg, from about 1 ng/kg to about 1 μg/kg, from about 1 ng/kg to about 500 ng/kg, from about 100 ng/kg to about 500 μg/kg, from about 100 ng/kg to about 100 μg/kg, from about 1 μg/kg to about 500 μg/kg, or from about 1 μg/kg to about 100 μg/kg. In each of these embodiments, dose/kg refers to the dose per kilogram of a patient's or animal's mass or body weight.

### Example 1

### Curcumin Reduces Neuronal Cell Injury and Effectively Promotes Functional Recovery in SCI Rats

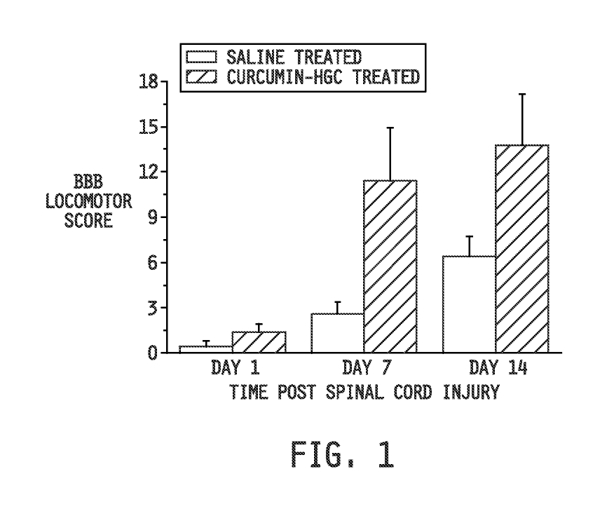

An in vitro study shows that curcumin is effective in reducing cell apoptosis in an H₂O₂-induced PC12 cell injury model. Curcumin-loaded, hydrophobically modified glycol chitosan (HGC) nanoparticles were administered to a group of five Long Evans rats at two hours after traumatic spinal cord injury (SCI). All the rats showed a significant functional recovery, as evidenced by an increase of Basso Beattie Bresnahan (BBB) locomotor rating score to an average value of 13.8 at day 14. In the control group treated with saline, the average BBB score was 6.4 at day 14 (see FIG. 1).

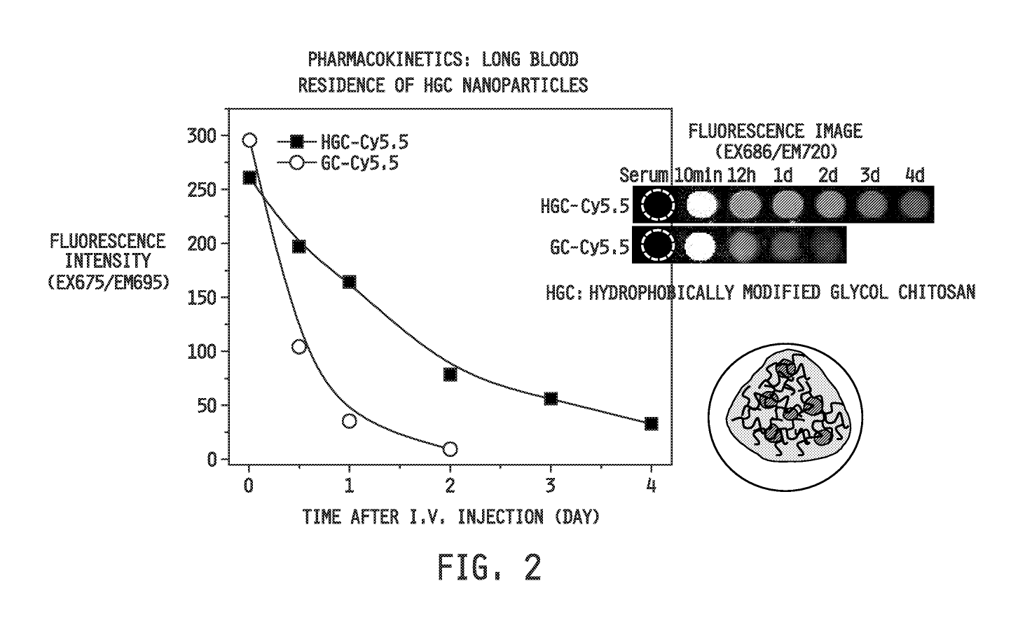

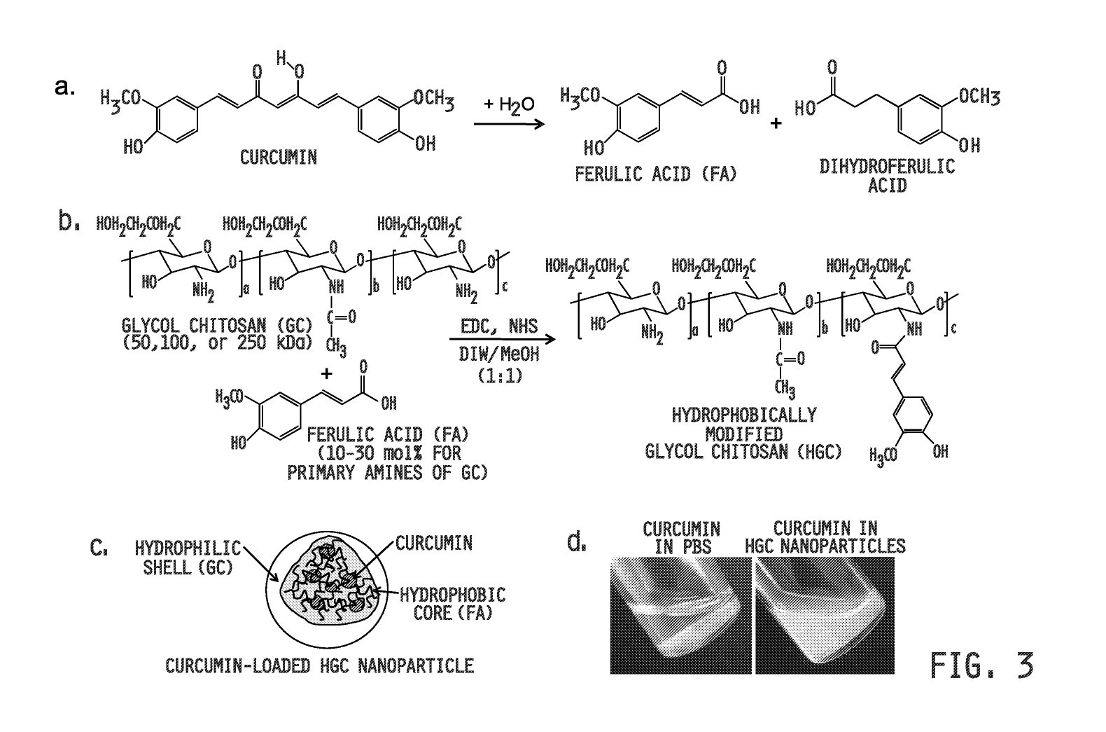

In a separate experiment, the HGC nanoparticles had a blood half-life time of 12 hours (see FIG. 2). The enhanced circulation time of the HGC nanoparticles ensures the delivery of the carrier and drug to the site of injury. These data show encouraging evidence that an extended therapeutic time window is achievable by using self-assembled nanostructure of amphiphilic polymer encapsulated with curcumin.

### Example 2

### Preparation and Characterization of Polymer Nanostructures

An effective way to extend the therapeutic time window of micelle treatment is to encapsulate an anti-inflammatory agent into the hydrophobic core of the micelle, so that both primary and secondary injuries will be targeted. In parallel, a separate study showed that mPEG-polyester micelles with different hydrophobic chains exhibited different efficiencies in restoration of compound action potential, indicating a critical role of the amphiphilic property in membrane repair. Thus, the hydrophobic core of the micelle is designed based on two factors: the loading efficiency of the anti-inflammatory agent and the membrane repair efficacy.

Most of anti-inflammatory drugs that have been applied to SCI treatment are steroids and derivatives such as glucocoticoid, methylprednisolone, sodium succinate, and naloxone. However, high-dose steroids have been shown to increase the risk of wound infections, pneumonia, sepsis and death in SCI patients due to respiratory complications. Non-steroidal anti-inflammatory drugs are usually enzyme specific or immune selective which requires fundamental discovery of selective enzymes and immune pathways.

Curcumin, isolated from turmeric in Curcuma longa as a traditional food ingredient, has unique properties. In pharmacologic studies, turmeric exhibits antitumor, anti-inflammatory, and anti-infectious activities with low toxicity. Specifically, curcumin has been shown to inhibit tumor necrosis factor (TNF), downregulates interleukin (IL)-1, IL-6, IL-8, and chemokines, increase the expression of intracellular glutathione, suppress lipid peroxidation, and play an antioxidant role through its ability to bind iron. Curcumin has been applied in diseases such as Alzheimer's disease, Parkinson's diseases, cancer, and others. A major challenge facing clinical application of curcumin is its rapid systemic elimination. Thus, a stable carrier delivering curcumin to the target tissue is needed.

mPEG-polyesters of various molecular weights is prepared using the dialysis method and load curcumin into the hydrophobic core of the micelle. The loading efficiency of curcumin and stability of curcumin-micelle complex in serum was characterized. In parallel to the use of block copolymers, glycol chitosan with the side chains modified with ferulic acid (FA) was synthesized. With an extended blood residence half-life, glycol chitosan nanoparticles have been widely used as carriers of anti-cancer drugs. Because FA is a product of curcumin hydrolysis, the modification is expected to not only introduce the amphiphilicity, but also enhance the loading efficiency of curcumin following the law of similar mutual solubility. Compared to mPEG-PDLLA, the amine groups in chitosan help attach the polymer to the negatively charged cell membrane, which facilitates insertion of the hydrophobic side chain into a lipid membrane as well as cellular uptake of curcumin.

A. Preparation of Self-Assembled mPEG-Polyester Micelles and Loading of Curcumin

The mPEG-PCL(poly ε-caprolactone), mPEG-PLGA (poly lactic-glycolic acid), and mPEG-PLA(poly lactic acid) were synthesized by ring opening polymerization (Liggins et al. (2002) Adv Drug Deliv Rev 54:191-202, incorporated herein by reference). The molecular weight of PLA, PCL, PLGA will be 4000, and the PEG will be 2000, the same as the molecular weight of mPEG-PDLLA used in the pilot study.

To test the membrane repair efficiency as a function of hydrophilic-lipophilic balance values, mPEG (2000)-PDLLA copolymers with different molecular weights of PDLLA (4000, 8000, 16000 Da) will be synthesized by ring opening polymerization of D,L-lactide. Different D,L lactic acid to methoxy PEG feed ratios will be used to prepare mPEG-PDLLA copolymers with varying degrees of D,L lactic acid polymerization. In all cases, micelles will be prepared by membrane dialysis. CMC will be measured by monitoring the fluorescence behavior of pyrene entrapped in the hydrophobic core of the micelle (Schild et al. (1991) Langmuir 7:665-671, incorporated herein by reference). The diameter of micelles will be determined by dynamic light scattering. The number average molecular weight of the hydrophobic block is measured using the proton peaks' intensity in ¹H NMR spectra recorded on a Varian Unity Inova 500NB spectrometer (Palo Alto, Calif.) operated at 500 MHz.

Curcumin is loaded into the core of mPEG-PDLLA micelles through hydrophobic interactions. The mPEG-PDLLA copolymer and curcumin dissolved in acetone or dimethyl sulfoxide (DMSO) are placed in a porous dialysis tubing (Spectra/Pro), followed by dialysis against 4 L of distilled water for more than 24 h at 25° C. Feed ratio of polymer-to-drug is varied to find maximum drug loading content and best loading efficiency. The resultant solution is frozen in a −80° C. freezer and dried using a freeze-dryer FD-5N (EYELA, Tokyo, Japan). Fresh curcumin-loaded micelles in solution are made at the day of application by dissolving the freeze-dried powder in a PBS solution using sonication.

### B. Characterization of Curcumin-Loaded Micelles

1. Micelle size and stability The micelle size is an important parameter which correlates with solubilizing efficiency and activities in the blood stream. The size of particles in the dried state is measured by transmission electron microscopy (TEM; Philips CM 10, 80 kV) (Lee et al. (2007) Biomacromolecules 8:202-208, incorporated herein by reference). The size of empty micelles or curcumin-loaded micelles in aqueous condition is measured by dynamic light scattering (DLS, PDLLS/Batch DLS instrument connected to PD2000 DLS detector, Precision Detectors). The stability of these micelles is determined by the changes of size as a function of time in both aqueous water and serum. Zeta potential showing the net charge of polymer micelles is measured by ZetaPALS (Brookhaven Instruments).

2. Drug Loading Amount and Efficacy

To quantify the enhanced solubility of curcumin in micelle carriers, drug loading amount and efficacy is measured. Drug loading amount is defined as the weight ratio of the loaded drug to the micelles. Drug loading efficiency is the percent ratio of the drug incorporated into the micelles to the initial amount of the drug used in the micellization. In brief, 1 mg freeze-dried curcumin-loaded micelles is dissolved in 1 mL DMSO so that micelles will be dissociated and curcumin will be released. The fluorescence of curcumin is measured spectrophotometrically at 427 nm using a UV spectrometry (Spectra Max M5, Molecular Devices). The drug loading amount and loading efficacy is calculated based on a set of standard samples containing predetermined amounts of curcumin.

3. Drug Release

After intravenous injection, curcumin will be released in blood where the lipophilic components (e.g. albumin) act as sink condition. To measure the release kinetics of curcumin, 2 mg/ml curcumin-loaded micelles are dispersed in a tightened dialysis bag and placed in a glass vial containing 40 mL PBS, pH 7.4, with 15% serum. The glass vial is shaken in a thermostatically water bath maintained at 37° C. during the study. Approximately 1.0 mL of release medium is taken at predetermined time intervals and the same volume of PBS/Serum is refreshed. Cumulative amount of released curcumin is measured spectrophotometrically in DMSO and the concentration of the released curcumin is calculated using standard curve of curcumin in DMSO. All experiments can be done in triplicate.

### Example 3

### Preparation and Characterization of HGC Nanoparticles

### A. Preparation of Curcumin-Loaded HGC Nanoparticles

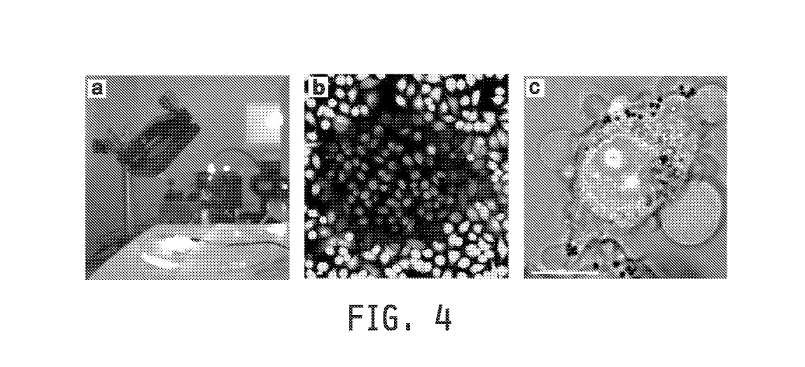

To synthesize hydrophobically modified glycol chitosan (HGC) nanoparticles with various molecular weights and hydrophobicities, glycol chitosan (GC) with different molecular weights (250, 100, and 50 kDa) will be prepared using an acidic degradation method. Then, the GC will be hydrophobically modified by conjugation with ferulic acid (FA) that is a product of curcumin hydrolysis (see FIG. 3a). By controlling the degree of conjugation of FA to GC, the hydrophobicity will be modulated. In detail, 50 mg of GC (50, 100, or 250 kDa in molecular weight) is dissolved in 15 ml deionized water, followed by dilution with methanol (15 ml), and mixing with FA (3.5, 7.0 and 10.5 mg, corresponding to 10, 20, and 30 mol % for the primary amines in GC). Conjugation of the carboxyl group in FA to the amine group in GC is initiated by adding EDC/NHS that is 1.5 fold molar excess of FA. The resulting solution is gently vortexed for 24 hours at room temperature, dialyzed (molecular cutoff=12 kDa) for 72 hours against excess water/methanol (1:4 volume ratio), followed by dialysis against deionized water, and the product is lyophilized to obtain HGC (see FIG. 3b).

The solvent evaporation method is used to encapsulate curcumin into the HGC nanoparticle. Both HGC and curcumin are dissolved in a co-solvent made of water and methanol (1:1 volume ratio). With the evaporation of methanol, HGC in the aqueous solution is hydrophobically self-assembled into nanoparticles composed of a hydrophilic shell and a hydrophobic core (see FIG. 3c). In detail, HGC (5 mg) is dissolved in deionzed water (2.5 ml), and mixed with curcumin solution (1.25 mg, 20 wt %) in methanol (2.5 ml). The methanol in the mixture solution is removed using a rotary evaporator. Preliminary data showed that FA-conjugated glycol chitosan effectively enhanced the solubility of curcumin in PBS solution (see FIG. 3d). No precipitation was observed over 1 month for the curcumin encapsulated in the HGC nanoparticles.

### B. Characterization of Curcumin-Loaded HGC Nanoparticles

The molecular weight of acid-degraded GC is measured by gel permeation chromatography (GPC). The degree of conjugation of FA to GC is determined by colloidal titration (Kwon et al. (2003) Langmuir 19:10188-10193, incorporated herein by reference) and UV absorbance of FA at 250-350 nm in DMSO. The loading amount and loading efficacy of curcumin in HGC will be examined using the same method as previously described. The measurement of physiochemical properties of curcumin-loaded HGC nanoparticles and curcumin release test will be conducted using methods previously described. In addition, x-ray diffraction is used to determine the degree of crystallization of curcumin inside the nanoparticle.

Two types of amphiphilic polymers are generated, PEG-polyester and FA-modified glycol chitosans of various molecular weights. A pool of polymeric nanostructures exhibiting different hydrophobicity and capable of curcumin loading will be ready for cellular, ex vivo and in vivo testing. In the following sections, “polymeric nanostructures” will be used to refer both PEG-polyester micelles and/or FA-modified HGC nanoparticles. In addition to the DLS measurement, Förster resonance energy transfer (FRET) spectroscopy is used to monitor the stability of micelles in serum. A FRET pair, DiIC₁₈₍₃₎ and DiOC₁₈₍₃₎, is loaded in micelles as previously described (Chen et al. (2008) Proc Natl Acad Sci USA 105:6596-6601, incorporated herein by reference). By monitoring the FRET efficiency, release of the core-loaded probes to surrounding medium is monitored in real time.

### Example 4

### Determination of Cell Rescue Efficiency of Polymeric Nanostructures Using a Photoacoustic Membrane Poration Model

The micelles and HGC nanoparticles prepared in Examples 2 and 3 are screened to identify the nanostructures that are able to rescue the injured cells over an extended time window and to better understand how hydrophobicity affects the polymer-membrane interactions. A photoacoustic membrane poration model is used to mimic the traumatic cell injury. The membrane sealing efficiency is quantified by imaging cellular uptake of fluorescently labeled dextrans of various molecular weights. The cells are assessed by apoptosis and necrosis assays, as well as inflammation markers.

### A. Photoacoustic Membrane Poration Model

A membrane poration model that involves femtosecond (fs) laser irradiation of gold nanorods targeted to the cell surface has been demonstrated (Tong et al. (2007) Adv Mater 19:3136-3141, incorporated herein by reference). In this model, the nanorod surface is conjugated with a positively charged peptide (octaarginine, R8) for attaching the nanorod to the negatively charged cell surface. Because of the size effect, i.e., the hydrodynamic diameter of these nanorods is c.a. 100 nm, the nanorods stay on the cell surface for at least one hour before entering the cells. Laser irradiation of the nanorods produces a photothermal effect via plasmonic absorption and relaxation of the optical energy into phonon energy inside the nanorods. The thermal expansion of the nanorods generates a burst acoustic (or mechanic) wave that compromises the integrity of the cell membrane. The poration of plasma membrane leads to an influx of Ca²⁺ into cells and a subsequent activation of calpain which degrades the cytosketon and causes blebbing of plasma membrane. The injured cells can be labeled by propidium iodide, a necrosis marker (see FIG. 4). This process highly mimics the damage of neuronal cell membrane after a trauma injury. Without laser irradiation, we have previously shown that R8-conjugated nanorods (R8-NRs) caused no toxicity to cells.

To use this model for screening of membrane repair agents, PC12 cells are grown, as to mimic neuronal cells, in a collagen coated 96-well plate and incubated with R8-NRs (O.D. 1, 10 μl) for 1 hour. The binding of R8-NRs on cell surface is confirmed by two-photon luminescence (TPL) imaging before laser irradiation. After washing with PBS, membrane poration is induced by laser irradiation with a fs Ti:sapphire laser (MaiTai HP, Spectra-Physics) having a pulse width of 130 fs and a repetition rate of 80 MHz. The laser is tuned to the wavelength of plasmon resonance peak of R8-NRs. The formation of pore on plasma membrane and the pore size are tested by quantifying the cellular uptake of dextran-FITC with different molecular weight (eg. 4 KDa, 10 KDa, 70 KDa). Dextran-FITC is added prior to irradiation. For each type of dextran-FITC, the irradiation condition (laser energy, irradiation time) is optimized to induce at least 80% of cells permeable. Cells are visualized using an Olympus FV1000 confocal microscope in Weldon School of Biomedical Engineering.

### B. Testing Methods

1. Cell Viability

Cell death is determined using a standard apoptosis kit (Invitrogen) including Alexa Fluor 680 annexin V to indicate early apoptosis and propidium iodide to label necrosis. A total of 5 μL of Alexa Fluor 680 annexin V and 1 μL of propidium iodide (100 μg/mL) are added to the cells after treatment or without treatment as a control as previously described (Tong et al. (2009) Nanomedicine 4:265-276, incorporated herein by reference). Independently, a MTT assay is also be performed to quantify the cell death. After laser irradiation and following the treatment, 10 μL MTT solution (5 mg/mL in PBS) is added to each well of the 96 well plate and incubated at 37° C. for 3 hours. After removing the medium, 200 μL DMSO is added to each well and the optical density is read at 570 nm using a spectrophotometer (SpectraMAX 190, Molecular Devices Corp., CA). Cell viability is assessed 24 hours post photoacoustic poration.

2. Cell Membrane Integrity

The sealing of cell plasma membrane is tested by adding dextran-rhodamine prior to irradiation and dextran-cy5.5 at different time points post-irradiation. Once the cell membrane is repaired, the uptake of dextran-cy5.5 is stopped. The percentage of cell rescue is calculated by (Nrho positive—Ncy5.5 positive)/Nrho positive, where N is the number of cells labeled by rhodamine or cy5.5. Images are taken by confocal microscope and the number of cells is counted by ImageJ software.

### 3. Intracellular Inflammation

Intracellular reactive oxygen species (ROS) is used as a marker of inflammation. Twenty four hours post-treatment, carboxy-H2DCFDA (Invitrogen) (a ROS indicator) is added to the cells and incubated for 30 minutes. Images are taken by confocal microscope. The intensity between treated and control groups is compared to characterize the amount of ROS.

4. Experimental Design

To determine the cell rescue efficiency, PC 12 cells are divided into four groups: group 1 containing cells with no photoacoustic poration, group 2 containing cells treated with curcumin-loaded polymeric nanostructures after photoacoustic poration, group 3 containing cells treated with curcumin-free polymeric nanostructures after photoacoustic poration, and group 4 containing cells treated with photoacoustic poration alone. To examine the effectiveness of polymeric nanostructures at different lag times between poration and administration, the nanostructures are added into the cell culture solutions at 15 minutes, 1 hour, 2 hours, and 6 hours post-photoacoustic poration in group 2 and group 3, respectively. Two-way ANOVA test is used to compare the efficiency of different treatments statistically.

Effective nanostructures are identified and the dose response is further examined to provide a reference of dose regimen for ex vivo and in vivo studies. As shown in a previous study (Shi et al. (2010)), the mPEG-PDLLA copolymers are effective as low as 3.3 μM when administrated to the spinal tissue, therefore, polymeric nanostructures with unimer concentration of 0.33 μM, 3.3 μM, 33 μM, and 330 μM will be applied to the cells cultured in the 96-well plate after photoacoustic poration.

Membrane sealing is believed herein to depend on the amphiphilic property of the polymer. A range of polymeric nanostructures may be identified that are able to seal the damaged membranes and also suppress the intracellular inflammation via the loaded curcumin. The optimal nanostructures should have good efficacy of cell rescue with a lag time of at least 2 hours. Because both charge and size affect the diffusion of molecules in a tissue environment, cellular-level effective nanostructures with different size and charge properties will be tested in Example 5.

An alternative method for membrane poration is by a laser-enabled analysis and processing (LEAP) apparatus available in Purdue University Bindley Bioscience Center.

### Example 5

### Determination of Functional and Morphological Response of Ex Vivo Spinal Cord Treated with the Nanoscale Repair Agents

The cellular study in Example 4 provides a means of fast screening of a large amount of candidate nanostructures. The tissue-level functional and morphological responses to these nanostructures are determined in this example. Spinal tissues are more compact and may not be readily assessable by polymeric nanostructures compared with cell culture condition. Functional measurements provide important selection criteria for further in vivo studies. As the example, isolated spinal cords from adult guinea pigs are compression injured, treated with the candidate nanostructures loaded with curcumin selected in Example 4, and assessed by electrophysiological measurement and morphological studies.

A. Recording of CAP with a Double Sucrose Gap Recording Chamber

Isolation of spinal cord white matter is performed following the procedures described in (Wang et al. (2005) Biophys J 89:581-591, incorporated herein by reference).

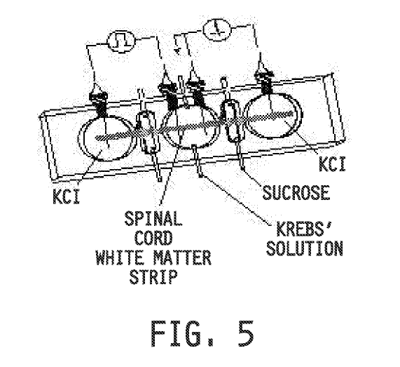

CAPs are recorded using a double sucrose gap recording chamber (see FIG. 5). A 4.0 cm long strip of isolated guinea pig spinal cord white matter is supported in the central compartment and continuously perfused with oxygenated Krebs' solution (˜2.0 ml/min) at 37° C. maintained in a water bath. The free ends of the spinal cord strip are carried through the sucrose gap channels to side compartments filled with isotonic (120 mM) potassium chloride. The white matter strip is sealed on either side of the sucrose gap channels, using fragments of plastic coverslip and a small amount of silicone grease to attach the coverslip to the walls of the channel and seal around the tissue. Isotonic sucrose solution (230 mM) is continuously running through the gap channels at a rate of 1.0 ml/min. The axons are stimulated and CAPs are recorded at opposite ends of the strip of white matter by silver/silver chloride wire electrodes positioned within the side chambers and the central bath. Stimuli, in the form of bipolar square pulses of 0.1 ms duration, are adjusted to the smallest amplitude that could produce a full action potential for each sample.

### B. Compression Injury and Treatment

The compression injury will be inflicted by a constant displacement of 5-30 sec compression of the spinal cord using modified forceps possessing a spacer until the CAP drops to 0 mV (Luo et al. (2002) J Neurochem 83:471-480, incorporated herein by reference). For local application of micelles, immediately after injury, the spinal cord white matter strips are kept in perfusing Krebs' solution at the speed of 2.0 ml/min. Then the perfusion is stopped and polymeric nanostructures are added gently to the Krebs' solution in the central compartment at 15 minutes, 1 hour, 2 hours, and 4 hours post compression injury, at a desired concentration determined by Example 4. Following the treatment for 10 minutes, the spinal cord strips are thoroughly rinsed with Krebs' solution. All the solutions are enriched with 95% O₂/5% CO₂ throughout the experiment.

C. Multimodal NLO Imaging to Monitor Ca²⁺ Entry into Axons

A multimodal NLO microscope has been developed that combined CARS and TPEF on the same platform (Chen et al. (2009) Opt Express 17:1282-1290, incorporated herein by reference). CARS imaging of myelin sheath is used to define the intra-axonal space. For monitoring calcium entry into axons, the spinal sample is pre-incubated in Ca²⁺-free Krebs' solution for 30 min, followed by Ca²⁺-free Krebs' solution with 40 μM Oregon Green 488 BAPTA-2 AM (Sigma) for 2 hours. After that, the control group of healthy spinal cords is incubated in normal oxygenated Krebs' with Ca²⁺ for 1 hour; the control group of injured spinal cords are compressed and then incubated in normal oxygenated Krebs' with Ca²⁺ for 1 hour; the nanostructure treated group is compressed and then incubated for 1 hour in oxygenated Krebs' solution supplemented with polymeric nanostructures at the concentration identified in Aim 2. TPEF signal of Oregon Green will be transmitted through two 520/40 bandpass filters (Ealing Catalog Inc.) and detected by an external photomultiplier tube (H7422-40, Hamamatsu). FluoView software (Olympus, Tokyo, Japan) will be used to merge TPEF and CARS images, and quantify TPEF intensities inside axons.

### D. Measurement of Anti-Inflammatory Response

The anti-inflammatory role of curcumin is tested by western blotting of IL-1 and caspase 3 level in homogenized spinal tissue. The role of curcumin in reducing oxidative stress is determined by measuring the extent of lipid peroxidation and the content of glutathione inside the injured tissue.

### E. Experimental Design

Spinal cord ventral white matter from adult female guinea pigs (350 to 500 g body wt) is used. Spinal cords are divided into three groups treated by curcumin-loaded mPEG-polyester micelle, curcumin-loaded HGC, and saline, respectively. mPEG-polyester micelles and HGCs are tested. The micelles and HGCs are administrated at 15 m minutes, 1 hour, 2 hours, and 4 hours post-SCI. These time points will yield a time-dependence curve for each nanostructure. For CAP measurement, 10 spinal cords with the length of 4.5 cm each are used to test each administration. Spinal cords of 1-cm segments are used for imaging experiments and measurement of anti-inflammatory response (n=5 per test).

The plasma membrane damage may also be determined using three molecules with different molecular weights: ethidium bromide (EB, MW 400 Da), horseradish peroxidase (HRP, MW 44 kDa, type VI) and lactate dehydrogenase (LDH, MW 140 kDa). EB and HRP are added to the solution and the uptake of EB and HRP through the membrane breach of the spinal tissue is monitored. The number of EB positive cells and HRP labeled axons are quantified. LDH is usually confined inside the cell since it is unable to pass through the intact membrane. Therefore, the leakage of this enzyme to the extracellular space is indicative of membrane disruption. To detect LDH release, the solution bathing the spinal tissue is collected at the end of each treatment. The spinal tissue is quickly homogenized and the residual tissue LDH will be assessed by a lactate dehydrogenase test kit (Sigma, Mo.).

Example 5 will determine the membrane sealing effect and anti-inflammatory effect of the polymeric nanostructures at the tissue level. The optimal nanostructures or nanoparticles should have good efficacy to facilitate the CAP restoration with a lag time of at least 2 hours, and can be used in Example 6.

### Example 6

### Determination of Anatomical and Functional Recoveries Mediated by Curcumin-Loaded Copolymer Micelles and HGC Nanoparticles Using a Contusion SCI Model

A prior study showed the effectiveness of mPEG-PDLLA micelles in restoring CAP of injured spinal cord white matter tissues at a concentration that is 10⁵ orders lower than PEG. Moreover, it has been shown that intravenously administrated mPEG-PDLLA micelles were able to significantly improve locomotor functions in a Long-Evans rat model of compression spinal cord injury (see FIG. 1).

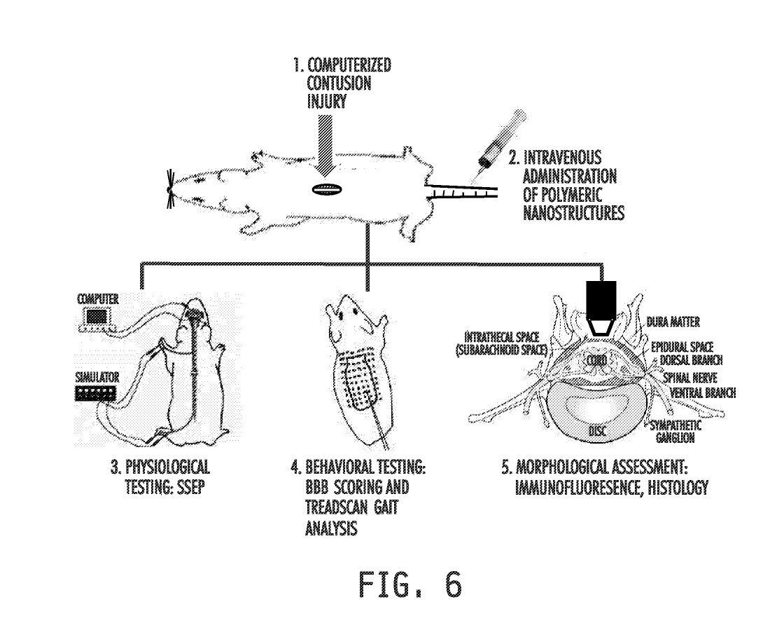

To determine the anatomical and functional recovery after SCI, mediated by mPEG-polyester nanostructures and/or HGC nanoparticles, polymeric nanostructures can be administered via tail vein in a clinically-relevant contusive injury model in adult rats, and the outcomes can be examined by using a combination of physiological, behavioral, and morphological assessments. The flowchart of the in vivo study is illustrated in FIG. 6, and the methods are detailed below.

### A. In Vivo Spinal Cord Injury Model

Computer controlled impact contusion is widely used by the SCI research community. Briefly, a moderate contusion injury can be induced by weight-drop of a 10 g rod from a height of 12.5 mm using a Multicenter Animal Spinal Cord Injury Study (MASCIS) spinal cord impactor. Detailed procedures are described in (Cao et al. (2005) Experimental Neurology 191:S3-S16; and Titsworth et al. (2009) Glia 57:1521-1537, both incorporated herein by reference).

### B. Bioavailability Assay

The polymeric nanostructures or the HGC nanoparticles can be delivered via tail vein or jugular vein injection. The curcumin-loaded nanostructures or nanoparticles are believed to penetrate through the damaged blood-spinal cord barrier (BSCB) and accumulate at the site of injury with high concentrations. To facilitate penetration, if necessary, the nanostructures or nanoparticles may be delivered intrathecally or by direct injection into the cord parenchyma.

Autofluorescent curcumin and cy5.5 labeled copolymers can be used for a bioavailability study. Organs including the spinal cord can be extracted at 24 hours after injection of nanostructures and are examined on Caliper IVIS Lumina II which has a spatial resolution of 50 μm. The biodistribution of the carrier and curcumin at cellular level can be observed using a confocal microscope.

For the bioavailability assay, mass spectrometry may also be used to determine the concentration of curcumin (MW 368) in each extracted organ using isotope-labeled curcumin as an external standard.

### C. Behavioral Testing

Effective restoration of the lost locomotor function can be a primary aim of this example in experimental SCI. The following tests can be performed to assess different aspects of SCI outcomes.

### D. Locomotor Score

A popular and standardized locomotor rating scale is the BBB locomotor rating scale (Basso et al. (1995) Journal of Neurotrauma 12:1-21) which was used in the MASCIS. Using the standard BBB paradigm, animals are first be pretrained to locomote in an open field that consists of a plastic pool approximately 90 cm in diameter with 7-10 cm-high walls. Two independent examiners study the locomotor ability of each test subject for approximately 4 minutes, and then rate the subject locomotion using a 21-point scale. Following the SCI and treatment by polymeric nanostructures, the animals can be subsequently tested beginning as early as 1 day post-treatment with repeated weekly testing routinely extending to 8 weeks post-treatment.

### E. TreadScan Gait Analysis

The TreadScan system measures the forced locomotion, which meets the needs for gait analysis of animals. Gait analysis allows highly sensitive, noninvasive detection and evaluation of many pathophysiological conditions occurring in SCI. The TreadScan system takes video of an animal, running on a transparent belt treadmill using a high-speed digital camera. The TreadScan system can reliably analyze the video, and determine various characteristic parameters including the stance time, the swing time, total stride time, stride length, foot contact area size, body-foot spacing distance, foot spacing distances, running speed, stride frequencies, foot coupling measures, and sciatic function index related measures such as foot print placement rotation angle with body and toe spread factors. TreadScan outputs the detailed results of these parameters into Microsoft Excel files and gives statistical results to meet research requirements.

### F. Neuronal Activity Monitoring by Electrophysiology

Somatosensory evoked potential (SSEP) (Kearse et al. (1993) Journal of Clinical Anesthesia 5:392-398; Hurlbert et al. (1993) J Neurotrauma 10:181-200, both incorporated herein by reference) can be used to evaluate the loss and recovery of electrophysiological conduction through the SCI. The electrophysiological measurements can be performed prior to laminectomy, immediately after compression, and weekly during the recovery period. The SSEP represents multisynapse afferent conduction through ascending long tract sensory columns and can be immediately eliminated by compression of the spinal cord between the sites of stimulation and recording. The stimulation of the tibial nerve of the hindlimb that produces ascending volleys of nerve impulses may be recorded at the contralateral sensory cortex of the brain. Each complete electrical record can be comprised of separate trains of 200 stimulations (<2 mA square wave, 200 μs duration at 3 Hz), offered by a Neuropak 8 stumulator/recorder (Nihon Kohden Inc., Tokyo, Japan) from subdermal needle electrodes placed on the skull evoked by bilateral simultaneous stimulation of the tibial nerve.

### G. Morphological Assessment

Morphological assessment using histology can provides the visual evidence of morphological change and recovery in axons, proteins and glial cell activity, which helps in-depth study of SCI pathogenesis and repair mechanism. The activities of astrocyte and immune cells can be investigated using immunohistochemistry. Details of these assays are described in the pilot study (Shi et al (2010)). Additionally, morphological test of myelin loss and intra-axonal spectrin breakdown can be performed to independently evaluate the recovery. The anti-inflammatory effects of curcumin can be examined by Western blotting of IL-1 and caspase 3 in the injured tissue.

### H. Assessment of Toxicity

To examine the safety of the nanostructures or nanoparticles, blood pressure and electrocardiogram can be measured before and after administration and subsequent animal body weight is monitored every other day. For complete blood counts (CBC), 1 ml of blood can be collected from jugular veins every 4 weeks after administration. At the end of locomotor function recovery study, a full gross necropsy examination can be performed. The weight of liver, spleen and kidney, as well as of any unusually sized organs, can be recorded. Tissues will be fixed in 10% neutral buffered formalin, processed routinely into paraffin, and 5-μm sections can be stained with haematoxylin and eosin. Liver, spleen, kidney, heart, lung, pancreas, urinary bladder, brain and spinal cord can be examined by light microscopy by a blinded rat veterinary pathologist. Urine samples can be collected every day in the first week post injury and once a week afterwards for analysis of pH, glucose, proteins.

### I. Experimental Design

Long-Evans rats can be used to examine the effectiveness of nanostructures or nanoparticles intravenously injected at various lag times after the injury. A total of seven groups of rats can be used to cover three lag times (2, 8, and 24 hours) and one control (saline injection at 2 hours). These time points can be selected based on the time course for primary injury. The dosage identified to be effective during tissue-level studies can be used. For monitoring locomotor function recovery, BBB scores can be recorded by two independent observers being blind to the treatment (n=15 per group). For bioavailability assays, Cy5.5-labeled nanostructures or nanoparticles can be administrated at three lag times (2, 8, and 24 hours) (n=5 per group). Acute and chronic toxicity of the polymeric nanostructures at the dose used for treatment can be assessed (n=10/group). For immuno-analysis, the animals can be sacrificed at 2 weeks after the treatment (n=5 per group). For Western blot assays of IL-1 and caspase 3, the animals can be sacrificed at 7 days after the treatment (n=5 per group).

This example can identify nanostructures or nanoparticles that effectively recover the SCI rats when administrated hours after SCI. A dose responsive curve can be established to determine the optimal concentration of the nanostructures or nanoparticles. The dose can be used for the subsequent determination of the therapeutic time window, which is important for the pre-clinical testing of therapeutic efficacies.

### Example 7

### Synthesis and Characterization of FA-GC/Curcumin Nanoparticles

### A. Methods

The pharmacokinetics of hydrophobically modified glycol chitosan (HGC) nanoparticles is believed to be dependent on the hydrophobicity of the polymer. Three different molar ratios of ferulic acid (FA) to glycol chitosan (GC) (i.e., 45, 90, and 180) was tested. In all cases, FA was coupled to GC in the presence of EDAC and NHS in 10 mMHEPES buffer (pH 7.2)/DMSO co-solvent. The resulting solution was stirred for 1 day at room temperature, dialyzed (molecular cutoff=12 kDa) for 3 days against excess water/methanol (1v:4v), followed by dialysis against distill water, and the product was lyophilized to obtain FA-GC conjugates. The degree of substitution, defined as the number of FA per one glycol chitosan chain, was determined by UV absorbance of FA at 316 nm in DMSO. FA-GC conjugates with three different degrees of substitution (5, 11, and 21 FAs per GC chain) were obtained.

The curcumin loading was based hydrophobic interactions of curcumin with FA. The curcumin was encapsulated into the FA-GC nanoparticles by a solvent evaporation method. Briefly, both FA-GC conjugates and curcumin (20 wt. %) were dissolved in a co-solvent made of water and methanol (1:1 volume ratio). After the evaporation of methanol under vacuum at 55° C., the FA-GC in the aqueous solution was self-assembled into nanoparticles.

The loading contents of curcumin in the nanoparticles were determined by UV absorbance of curcumin at 430 nm in DMSO. A larger curcumin loading efficiency was demonstrated at a higher degree of FA substitution (see Table 1).

TABLE 1
Curcumin loading efficacy as a function of FA substitution degree

| Sample | Curcumin content (wt. %) |
|---|---|
| GC | 4.34 |
| GC-FA (DS = 5) | 14.26 |
| GC-FA (DS = 11) | 15.54 |
| GC-FA (DS = 21) | 17.62 |

DS: degree of substitution, indicated by the number of FA units per GC chain

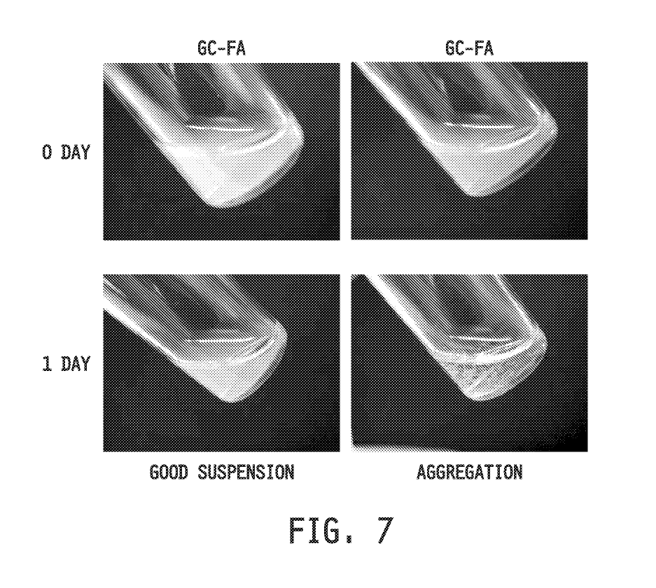

For the FA-GC nanoparticles with degree of substitution (DS)=21, the loaded curcumin precipitated after 1 day incubation in PBS (see FIG. 7). In contrast, the FA-GC with DS=11 was capable of stably encapsulating curcumin.

To label Cy5.5 to FA-GC polymer, 1 wt % hydroxysuccinimide ester of Cy5.5 was dissolved in DMSO and mixed with FA-GC solution. The reaction was performed at room temperature in the dark for 6 hours. Byproducts and unreacted Cy5.5 molecules were removed over a period of two days by dialysis (molecular weight=12 kDa) against distilled water, and the resulting product was lyophilized. The amount of Cy5.5 in the FA-GC was confirmed as 0.7 wt %, as determined by absorbance at 690 nm in DMSO. Curcumin was loaded to FA-GC(-Cy5.5) using the same method described above.

For biodistribution testing, the nanoparticles were administered to Long-Evans rats after contusion of the spinal cord. Tissue specimens including the injured spinal cords were harvested and homogenized at 1 hour post-injection. After adding warfarin (0.5 ppm) to the resultant solution, curcumin in the tissues was extracted by acetone. To quantify the concentration of curcumin in tissues, paper spray MS was performed.

Curcumin-loaded FA-GC nanoparticles were formed in PBS buffer (pH 7.4) by sonicated using a probe-type sonifier. Nanoparticle sizes and polydispersity (μ₂/Γ²) were determined using dynamic light scattering (DLS, 90Plus, Brookhaven Instruments Co., NY) at 633 nm and 25° C. The morphology of the nanoparticles in distilled water (1 mg/ml) was observed using transmission electron microscopy (TEM, CM 200 electron microscope, Philips). The surface charge in distilled water was determined using a zeta potential analyzer (ZetaPlus, Brookhaven Instruments Co., NY).

Confocal fluorescence images were obtained FV1000 confocal system (Olympus, Tokyo, Japan) equipped with Argon (488 nm) and HeNe (633 nm) lasers and 60X/1.2 NA water objective. Curcumin and FA-GC(-Cy5.5) images were acquired with 488 nm and 633 mm excitations, respectively.

For stability testing, curcumin and the nanoparticles were dispersed in PBS (pH 7.4) and incubated at room temperature. The solutions were monitored for one month.

### B. Results

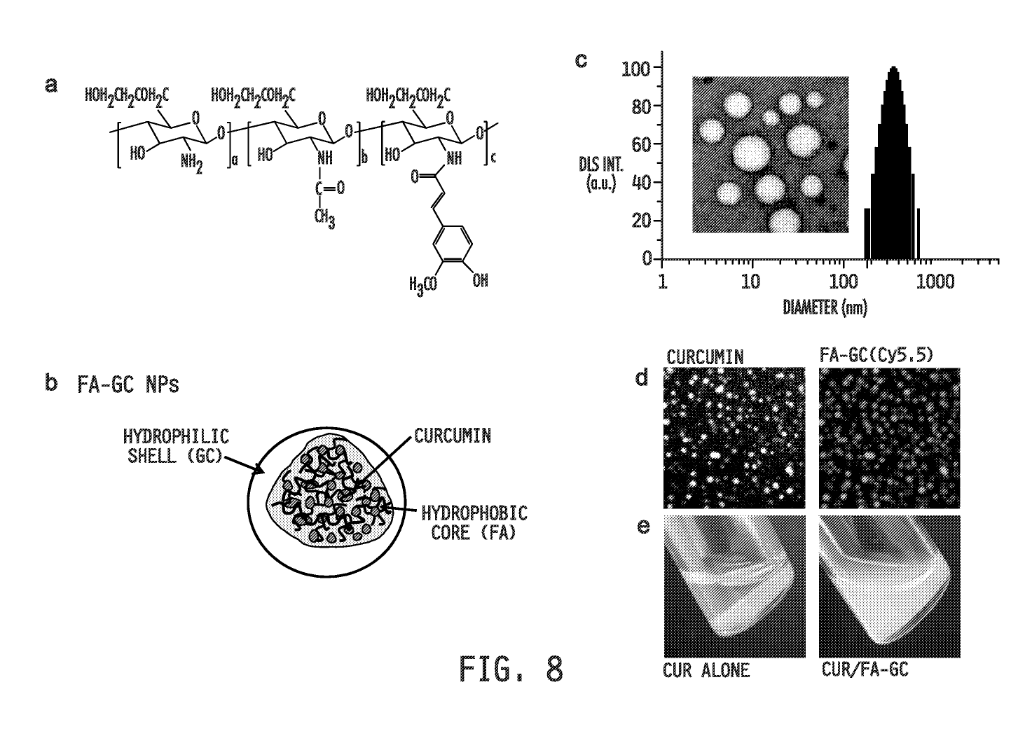

Glycol chitosan (GC, MW 250 kDa) was chemically conjugated with ferulic acid (FA), a product of curcumin hydrolysis (see FIG. 8(a)), to maximize the curcumin loading efficiency. An encapsulation efficacy of 15.54 wt % curcumin was achieved via optimization of the FA conjugation degree (see Table 1). By transmission electron microscopy (TEM) and dynamic light scattering (DLS), the average diameter of the cucumin-loaded GC-FA nanoparticles (see FIG. 8(b)) were determined to be 320 nm (see FIG. 8(c)). The polydispersity value (0.207) indicated a narrow size distribution of the nanoparticles. The zeta potential was measured to be 19.5 mV, indicating a positively charged surface of the nanoparticles. Co-localization of fluorescence signals from curcumin (see FIG. 8(d), left, green) and Cy5.5-labeled FA-GC (see FIG. 8(d), right, red) evidenced the encapsulation of curcumin into the nanoparticles. No precipitation was observed over one month for the curcumin present in FA-GC (see FIG. 8(e) and FIG. 7).

### Example 8

### Pharmacokinetics and Bio-Distribution of FA-GC/Curcumin

### A. Methods

Cy5.5-labeled FA-GC nanoparticles comprising cucurmin (5 mg/l ml in saline) or curcumin (0.77 mg/1 ml in saline with 0.1 (v/v) % Tween20) was intravenously injected through the jugular vein of rats at 2 hours post-contusive injury (n=3). Blood samples (100 μl) were drawn through the jugular vein at determined times. The blood (50 μl) was mixed with 5 μl K3 EDTA as an anticoagulation agent and warfarin (20 ng/4 μl, 0.5 ppm) as an internal standard for mass spectrometry analysis. To extract curcumin, acetone (150 μl) was added to the solution and vortexed for 10 minutes. The resulting solution was centrifuged (rpm 5000, 10 min), and the supernatant was stored at −20° C. until mass spectrometry analysis. To obtain a calibration curve for quantitative analysis, curcumin in rat blood with different concentrations (0-50 ppm) was prepared, and then curcumin was extracted by the same method described above.

For biodistribution testing of curcumin, the nanoparticles or curcumin/Tween20 with the same dose of the pharamcokentics study was administrated to Long-Evans rats (n=3) at 2 hours after contusion of the spinal cord. Tissue specimens including the injured spinal cord were harvested at 1 hour post-injection, and the tissues was homogenized using a grander. After adding warfain (20 ng/4 μl, 0.5 ppm) to the tissue solution (50 μl), curcumin was extracted by adding acetone (150 μl).

To determine the pharmacokinetics and bio-distribution of curcumin, paper spray mass spectrometry was employed. Paper spray mass spectrometry analysis was performed using a TSQ Quantum, LTQ ion trap, and ExactiveOrbitrap mass spectrometer.

The blood samples were collected at determined time points using the anticoagulant warfarin. After adding warfarin (0.5 ppm), curcumin in the blood was extracted by mixing with acetone to dissociate the curcumin-albumin complex. The resulting solution was loaded on a chromatography paper. After dropping 10 μl of methanol to the blood spot, the components in the blood were sequentially ionized by applying a DC voltage.

The pharmacokinetics and bio-distribution of FA-GC were determined by a fluorescence-based analysis. Half of the blood sample (50 μl) collected in the pharmacokinetics study of curcumin was used for the detection of Cy5.5 in the blood of rats (n=3). GC(-Cy5.5) (5 mg/1 ml in saline) as a control group was intravenously administrated to the rats (n=3) at 2 hours post-injury and then the blood was drawn by the same method as described above. At 1 day post-injection of curcumin-loaded FA-GC(-Cy5.5) to rats, the rats were sacrificed via transcardial perfusion with saline and the tissues then were harvested. The fluorescence intensity of Cy5.5 labeled to FA-GC polymer in blood and tissue samples was measured and visualized by a fluorescence spectrometer (SpectraMax M5, Molecular Devices, CA) with excitation at 675 nm and emission at 695 nm and IVIS Lumina (Caliper Life Sciences, Inc., MA) with excitation at 640 nm and emission at 695-770 nm. The quantitative analysis for the bio-distribution of FA-GC polymer was performed using the Living Imaging Software (Caliper Life Sciences, Inc., MA).

### B. Results

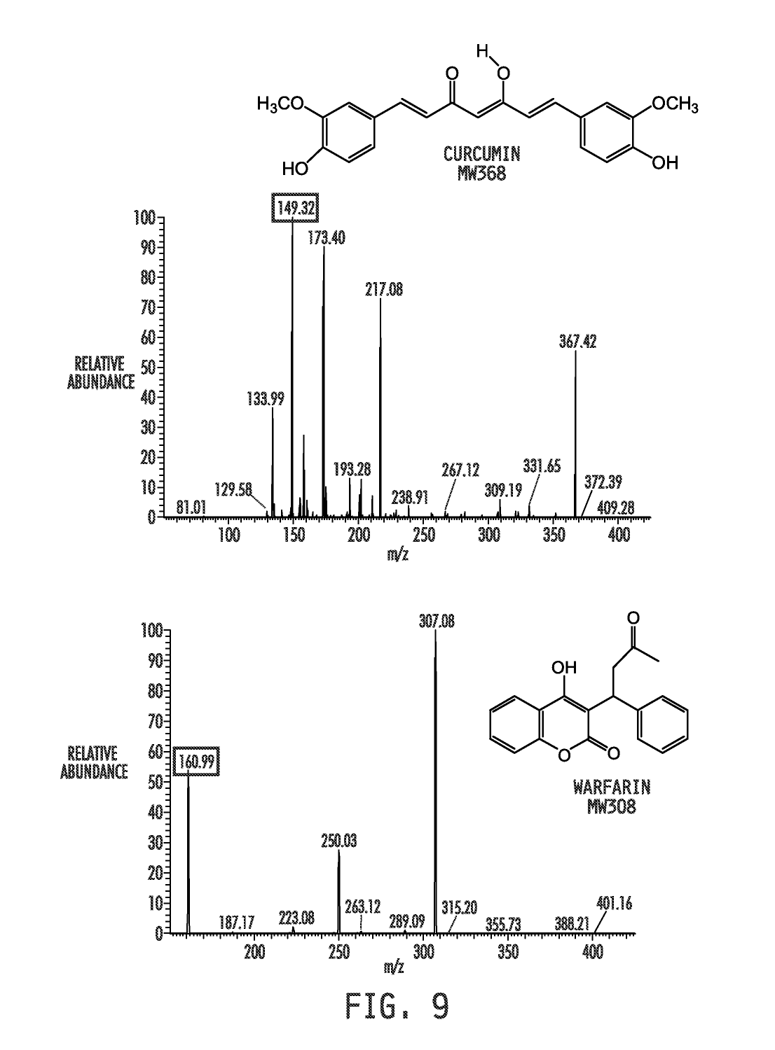

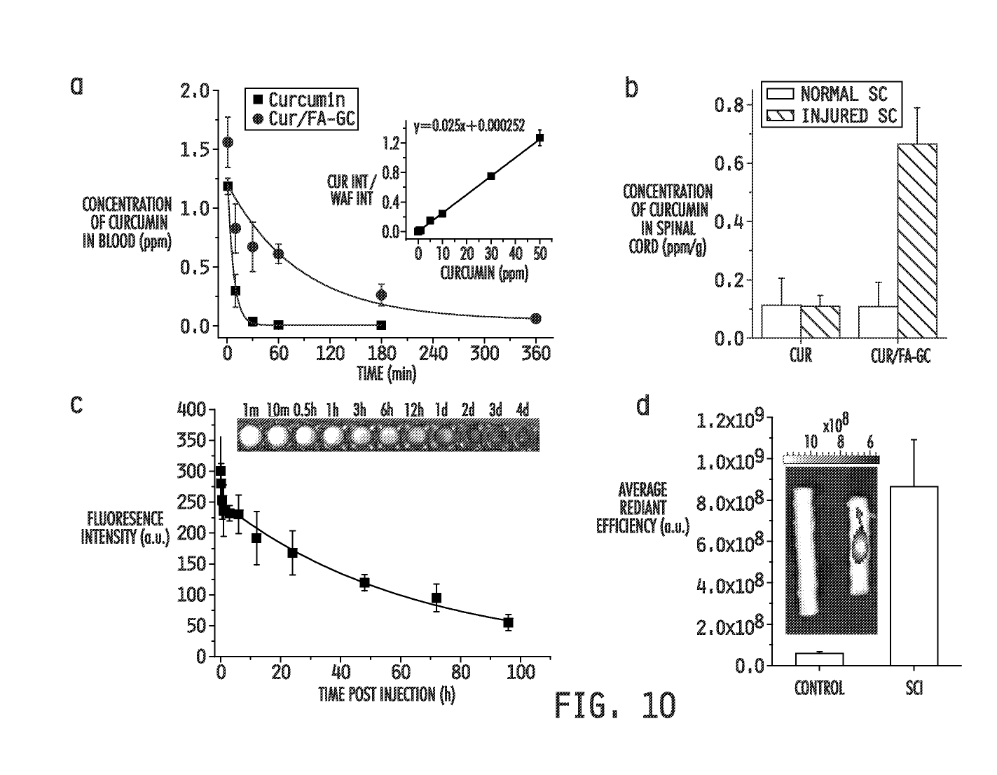

Following injection of Cy5.5-labeled FA-GC nanoparticles, curcumin and warfarin were detected by their ionized fragments (m/z=149 for curcumin, m/z=161 for warfarin) in the mass spectra (see FIG. 9). The concentration of curcumin was obtained by using a calibration curve derived from the ratio between mass intensities of curcumin and warfarin (see FIG. 10(a), insert). To determine whether our formulation could extend the blood retention time of curcumin, we compared the plasma concentration of curcumin between the GC-FA group and the control group in which the Tween20 surfactant was used as solubilizer of curcumin. Using the one-compartment model, the half-time of curcumin in the blood for the Tween20 group and the FA-GC group were measured to be 6 minutes and 36 minutes, respectively.

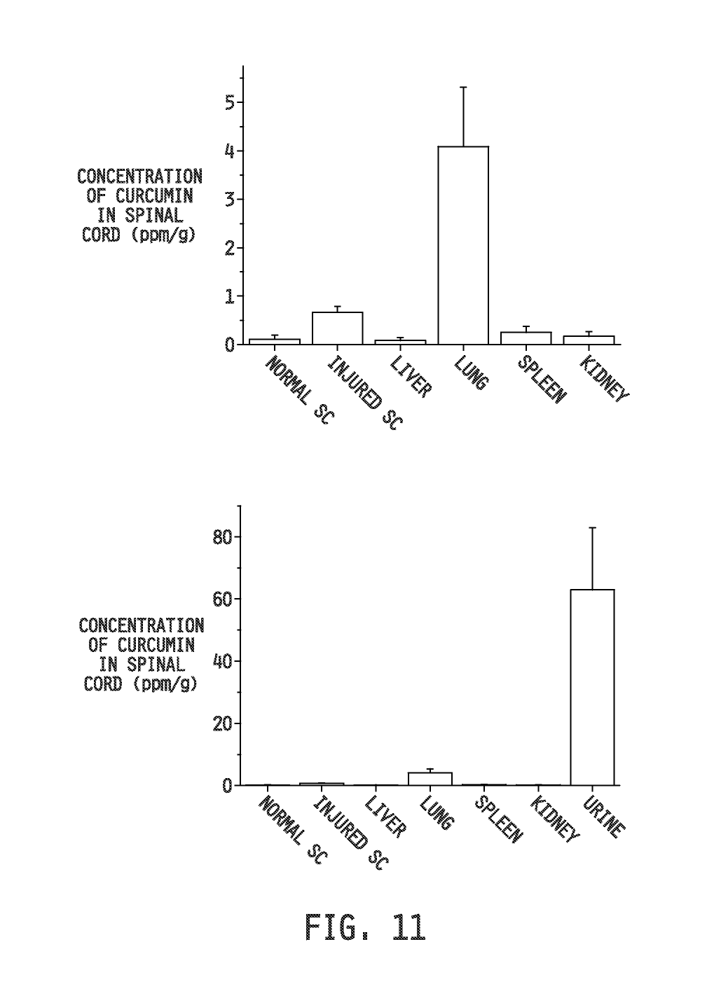

Biodistribution of curcumin was also studied by mass spectrometry. It was determined that curcumin in FA-GC nanoparticles mostly eliminated through the kidney (see FIG. 11). Importantly, the FA-GC group demonstrated 6.6 times higher concentration of curcumin in the injured cord compared to the normal cord (see FIG. 10(b)). In contrast, no difference was found between normal and injured cords for the Tween20 group (see FIG. 10(b)).

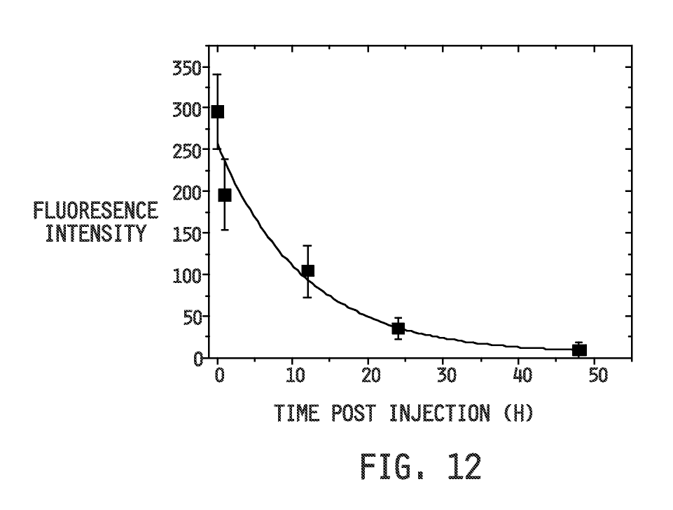

In determination of the blood retention time of GC polymers, the FA-GC exhibited a long blood retention time with a half-life of 20 hours determined by the one-compartment model (see FIG. 10(c)). In comparison, the non-modified GC showed a half-life of 6 hours (see FIG. 12).

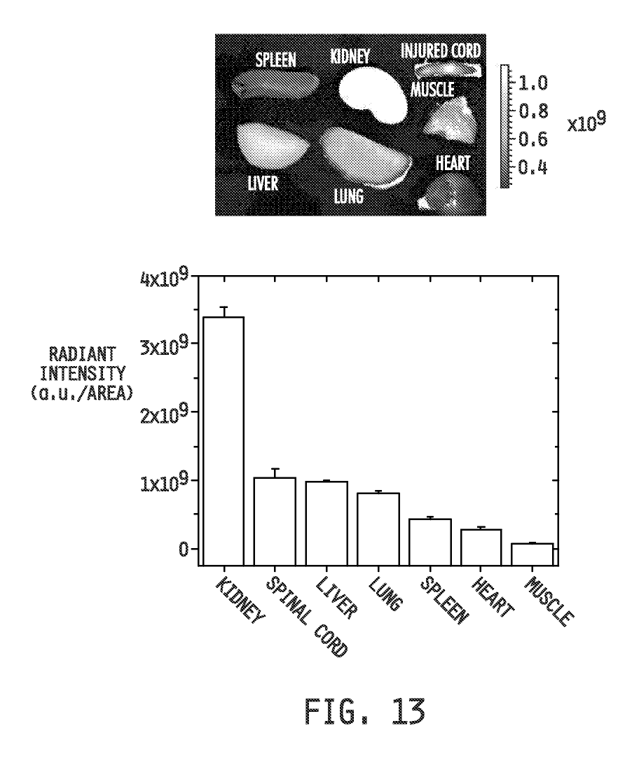

For the biodistribution assessment, main organs were harvested at 1 day after injection and the amount of Cy5.5 fluorescence was quantified by an IVIS instrument. The fluorescence intensity at the injured spinal cord was significantly higher than other organs (see FIG. 13), except for the kidney. Moreover, the strong signal was observed only at the lesion site of the spinal cord (see FIG. 10(d)). Collectively these data demonstrate that the hydrophobic modification of GC with FA allows for the prolonged circulation of the polymer and enhanced delivery of both polymer and curcumin to the injury site.

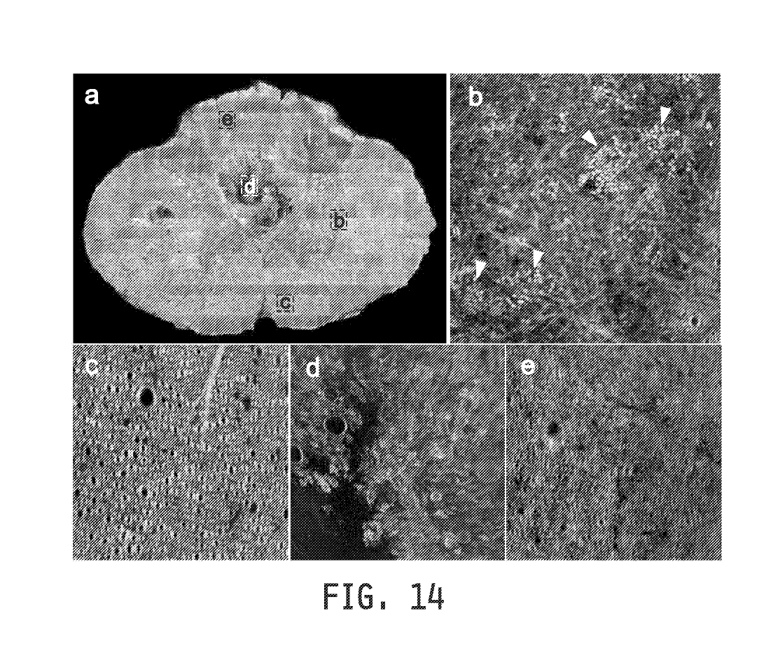

The distribution of FA-GC was further determined at single cell level using a multimodal nonlinear optical microscope that allows stimulated Raman scattering (SRS) imaging of membranes (green) and two-photon excitation fluorescence (TPEF) imaging of Cy5.5-labeled FA-GC (red). The polymers were found in both the injured white matter and the injured gray matter. Importantly, a strong fluorescence signal was found inside the gray matter that is highly vulnerable to a contusive injury, indicated by the formation of cavities (see FIG. 14(a)). High magnification SRS image of the gray matter showed clots of red blood cells (see FIG. 14(d), white arrows). The myelin sheath in the anterior white matter was highly convoluted exhibited (see FIG. 14(e)) while the myelin sheath in posterior white matter and near central canal showed irregular morphology (see FIGS. 14(b) and 14(c)). Together these results suggest the targeting of injured spinal cord by the FA-GC nanoparticles.

### Example 9

### In Vitro Model

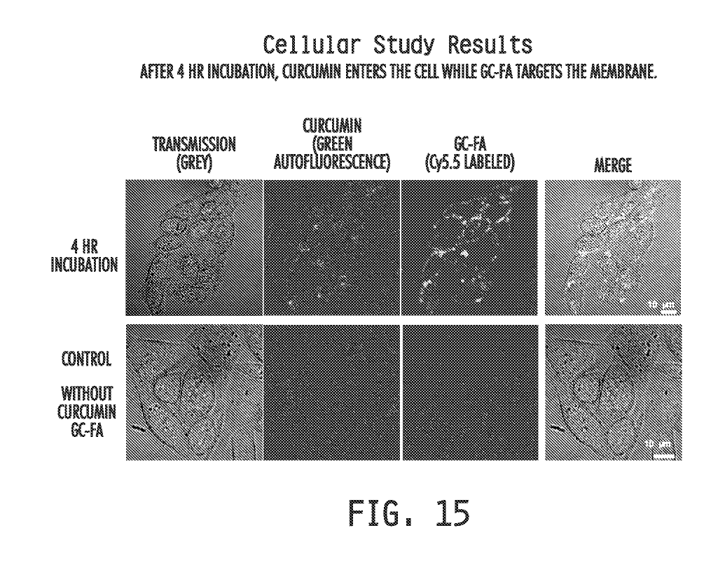

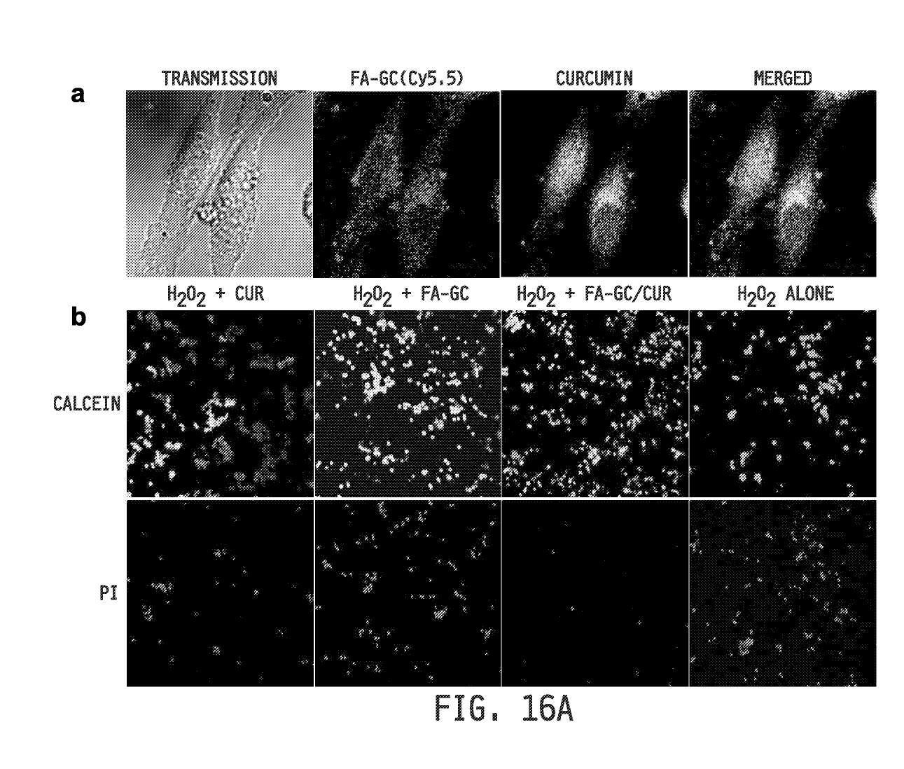

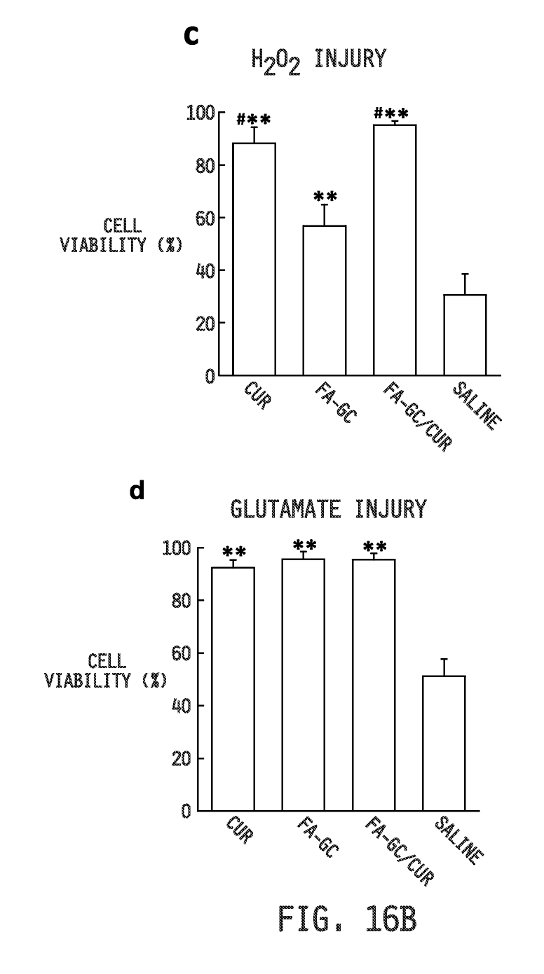

PC12 cells were used as a simple model for neuronal cells to evaluate the neuroprotective effect of the nanoparticles (as shown previously in FIG. 15, after a 4 hour incubation with GC-FA nanoparticles, curcumin enters cells and GC-FA targets the cell membrane). In this example, PC12 cells were incubated for 4 hours with curcumin-loaded GC(-Cy5.5)—FA nanoparticles. Thereafter, the cell membrane attachment of GC-FA and cellular internalization of curcumin were shown by confocal imaging (see FIG. 16(a)).

Because oxidative stress and glutamate excitotoxicity are two main different pathologies after spinal cord injury [x], the neuroprotective effects of the nanoparticles were further assessed using hydrogen peroxide (H₂O₂) and glutamate-injured PC12 cells. After incubating the cells with FA-GC/curcumin, FA-GC, or curcumin for 4 hours, cell viability was measured by calcein and propidiumlodide (PI) double staining.

Treatment with 0.2 mg/ml GC-FA/curcumin significantly reduced the number of PI stained cell (see FIG. 16(b)). GC-FA/curcumin treatment increased the survival rate from 20% to 95%, while GC-FA alone helped rescue the cells by 55% (see FIG. 16(c)). In the glutamate damage model, all three treatments significantly protected PC12 cells (see FIG. 16(d)). Together, these results suggest that the nanoparticles could effectively protect PC12 cells from H₂O₂ and glutamate injuries.

### Example 10

### In Vivo Spinal Cord Injury Model and FA-GC/Curcumin Administration

### A. Methods

All protocols for this example were approved by the Purdue Animal Care and Use Committee. Adult Long-Evans rats were anesthetized using 90 mg/kg ketamine and 5 mg/kg xylazine. A T10 laminectomy was performed to expose the underlying thoracic spinal cord segment(s). Spinal cord contusion injury was produced using a weight-drop device developed at New York University (Tcuner, 1992) and protocol developed by a multicenter consortium (Basso et al., 1996). The exposed dorsal surface of the cord was subjected to weight-drop impact using a 10 g rod (2.5 mm in diameter) dropped from a height of 12.5 mm. After the injury, the muscles and skin were closed in layers, and rats were placed on a heating pad to maintain the body temperature of the rats until they awake. The analgesic buprenorphine (0.05-0.10 mg/kg) was every 12 hours through subcutaneous injection during anaesthesia recovery and for the first 3 days post-surgery for pain management post-operation.

Rats were randomly divided into 4 administration groups for comparison: 1 ml FA-GC/curcumin (5 mg/ml in saline; n=10); 1 ml FA-GC alone (4 mg/ml in saline; n=8); 1 ml methylprednisolone sodium succinate (MPSS, 30 mg/kg; n=5); or an isovolumetric dose of saline (n=10). Treatments were administrated 2 hours post-injury by intravenous jugular vein injection. Manual bladder expression was carried out 3 times daily until reflex bladder emptying was established.

The locomotor recovery was assessed using the Basso Beattie Bresnahan (BBB) locomotor rating score. The test was conducted by two independently and made an agreement on the score before the scores were finalized. The BBB score was recorded at day 1, 7, 14, 21, 28 post-surgery.

### B. Results

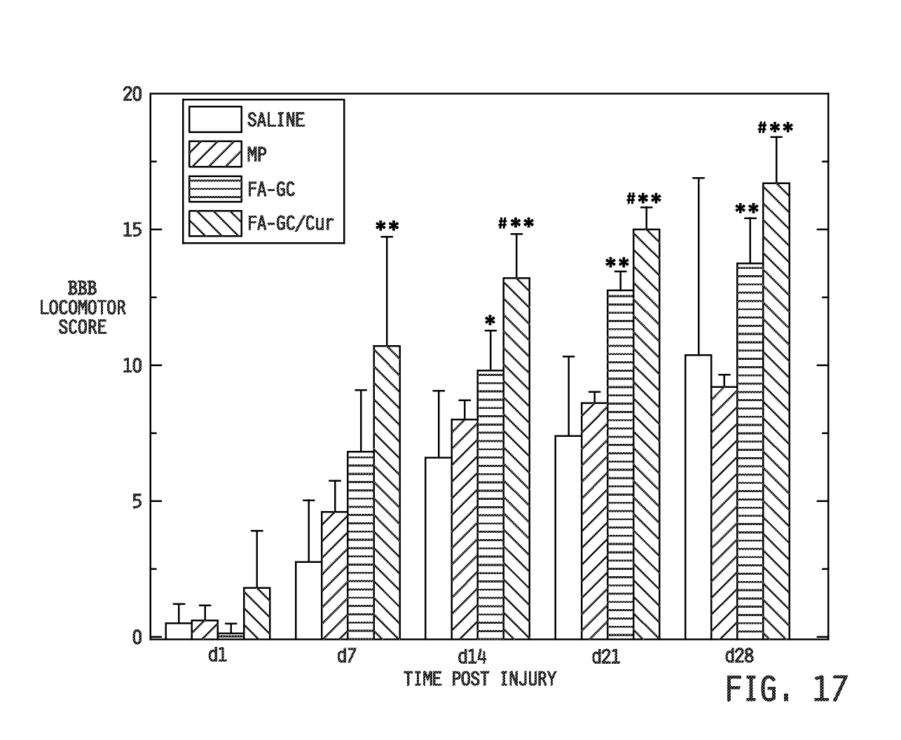

The recovery of locomotor function was evaluated and the results are shown in FIG. 17. Significant differences were found at day 7 and over the following 3 weeks between FA-GC/curcumin treated and MP treated rats. At day 28, the FA-GC/curcumin group was significantly better than the MP group by 6.3 points. Surprisingly, the FA-GC alone group also showed significantly higher score compared to saline control animals at day 14 and the following 2 weeks.

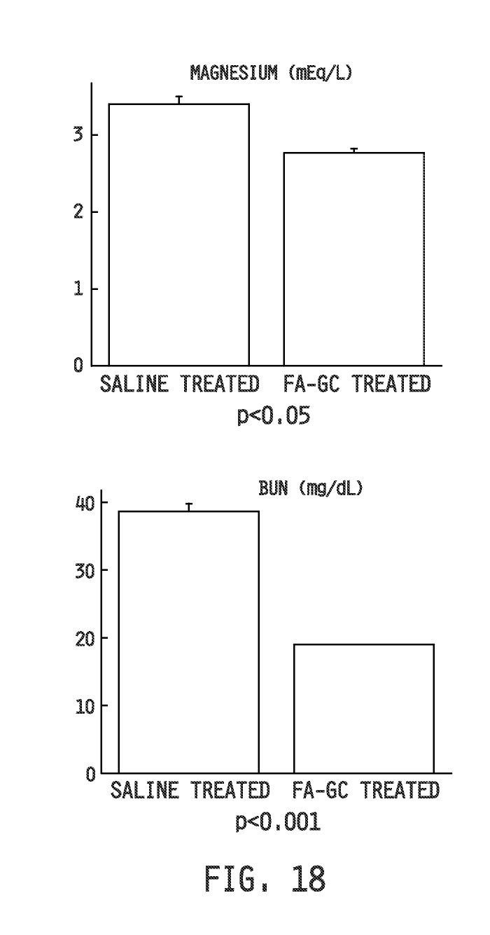

Blood and urine tests were also evaluated in an attempt to understand the repair mechanism. As shown in Table 2, levels of magnesium and BUN, two important kidney damage indicators, were significantly reduced after FA-GC treatment (see FIG. 18).

TABLE 2
Blood Test Results

| | | Total | | | | |
|---|---|---|---|---|---|---|
| | | Protein | BUN | | Calcium | Magnesium |
| | | g/dL | Mg/dL | Creatinine | Mg/dL | mEq/L |
| Saline | Rat 1 | 5.6 | 38 | 0.5 | 10.7 | 3.5 |
| treated | Rat 2 | 5.4 | 38 | 0.4 | 10.7 | 3.4 |
| | Rat 3 | 6.7 | 40 | 0.7 | 10.7 | 3.3 |
| FA-GC | Rat 4 | 6.5 | 19 | 0.4 | 10.7 | 2.7 |
| treated | Rat 5 | 7.4 | 19 | 0.6 | 11.1 | 2.8 |
| | Rat 6 | 6.3 | 19 | 0.4 | 10.4 | 2.8 |

| | | | | | |
|---|---|---|---|---|---|
| Normal range: | 5.6-7.6 | 8.5-22.7 | 0.2-0.8 | 5.3-13 | 1.5-2.5 |

In addition, FA-GC treatment also reduced the amount of white blood cells in urine (see Table 3).

TABLE 3
Urine test results

| | | Appear- | | | | Occult |
|---|---|---|---|---|---|---|
| | | ance | Color | Protein | WBC/HPF | Blood |
| Saline | Rat 1 | Turbid | Dark Yellow | 3⁺ | 0-1 | 2⁺ |
| treated | Rat 2 | Turbid | Dark Yellow | 3⁺ | 2-3 | 3⁺ |
| | Rat 3 | Turbid | Light Brown | 3⁺ | 2-3 | 3⁺ |
| FA-GC | Rat 4 | Cloudy | Yellow | 1⁺ | 0-1 | Negative |
| treated | Rat 5 | Cloudy | Light Yellow | Trace | None | Negative |
| | Rat 6 | Turbid | Dark Yellow | 2⁺ | None | 3⁺ |

### Example 11

### Spinal Cord Tissue Preparation and Histological Analysis of Spinal Cord Tissue Reactivity

### A. Methods

Tissue loss and cellular response were also evaluated between the FA-GC/Curcumin treated group and the saline control group. Four weeks post-injury, rats as described in Example 10 were anesthetized and transcardially exsanguinated with 150 ml physiological saline followed by fixation with 300 ml of ice-cold 4% paraformaldehyde in 0.01 M PBS (PH 7.4). A 1.5-cm thoracic Spinal cord segment at the lesion center were carefully dissected and then post-fixed overnight in 4% paraformaldehyde in 0.01 M PBS (PH 7.4), and transferred to 30% sucrose in 0.01 M PBS (pH 7.4). The cord segments were embedded in tissue-embedding medium, and 30-1 μm sagittal sections were cut on a freezing microtome and mounted onto glass slides.

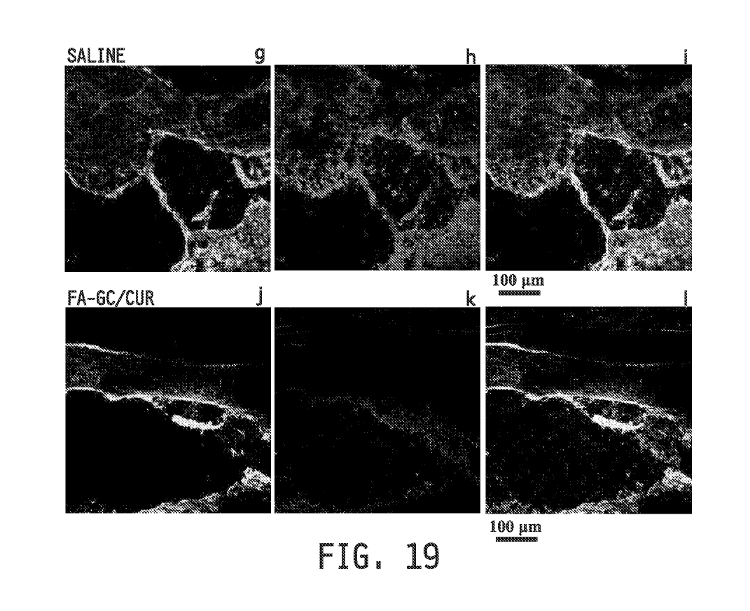

For immunofluorescence staining, the sections were permeabilized and blocked with 0.3% Triton X-100/10% normal goat serum (NGS) in 0.01 M PBS (pH 7.4) for 30 minutes, and primary antibodies were then applied to the sections overnight at 4° C. Glia fibrillary acidic protein (GFAP, diluted 1:220, Abcam) and ED-1 (diluted 1:50; Millipore, St, Charles, Mo., USA) were used as the primary antibody to identify astrocyte and macrophage/activated microglia (see FIG. 19). The sections were incubated the following day for 2 hours at room temperature with secondary antibodies (Alexa Fluor 488, Invitrogen; Cy3, Invitrogen), and were then washed, mounted, and examined using an Olympus IX70 confocal microscope equipped with a Fluo View program. The cavity volume, GFAP, and fluorescence intensity were measured using Image J.

### B. Results

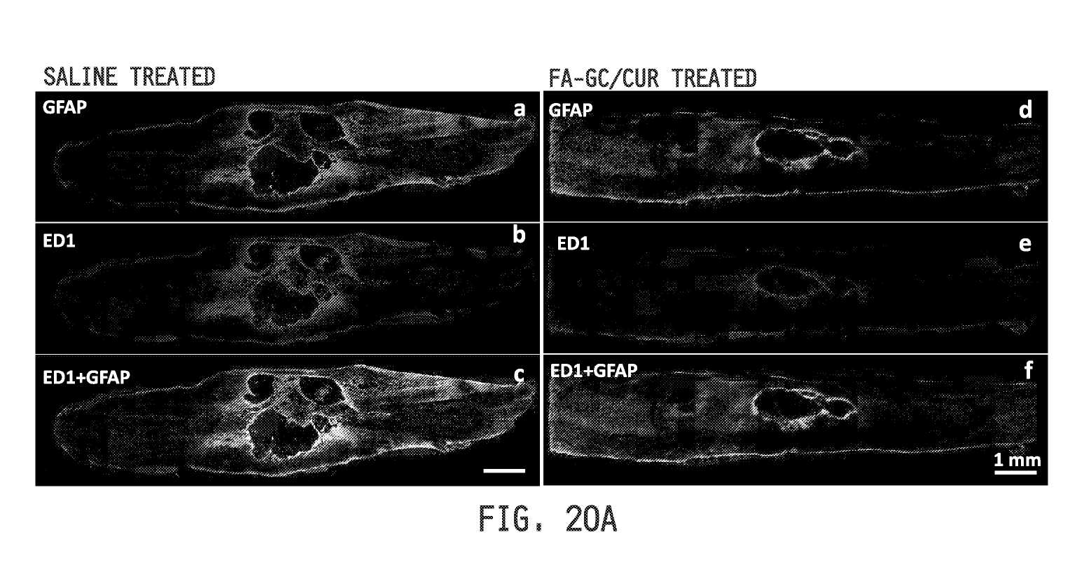

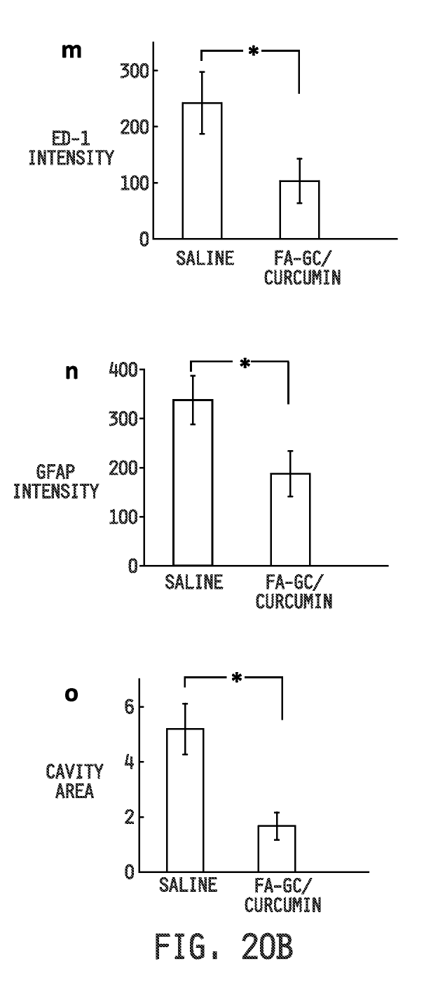

The cavity area indicated by astrocyte boundary is shown in FIG. 20(a) and FIG. 20(d), and the activated astrocytes and activated microglia are shown by the fluorescence of GFAP and ED-1 in the epicenter of the lesion (see FIG. 20(b) and FIG. 20(e)). FIG. 20(o) shows that the cavity area significantly decreased in FA-GC/curcumin treated group (1.67±0.5 mm²) compared to the saline control group (5.19±0.92 mm²). FIG. 20(m) and FIG. 20(n) show that the GFAP and ED-1 fluorescence significantly reduced in FA-GC/curcumin treated group compare to saline treated group (187.38±46.37 v.s. 339.37±49.47 for GFAP, 103.20±39.67 v.s. 242.35±55.38 for ED-1).

The animals with spinal cord injury but with saline treatment showed an obvious cavity in the white matter on the dorsal side. In contrast, treatment with FA-GC effectively mitigated the white matter loss.

### Example 12

### Nonlinear Optical Imaging of Spinal Tissues

The injured spinal cord tissue harvested in the biodistribution study of FA-GC was cross-sectioned at 200 μm thickness using an oscillating tissue slicer (Electron Microscopy Sciences, Inc., PA). For the SRL imaging, a Ti:sapphire laser (Chameleon Vision, Coherent) of 140 fs pulse duration, 80 MHz repetition rate was tuned at 830 nm to pump an optical parametric oscillator (OPO, APE compact OPO, Coherent). Based on the C-H molecular vibration, the OPO provided the Stokes beam at ˜1090 nm, and then collinearly combined with the pump beam and sent to a laser scanning microscope (BX51, Olympus). The pump and Stokes beam were then focused into the sample using a water immersion objective lens (XLPlan N 25X, NA 1.05, Olympus). The forward SRL signal was collected by an oil condenser (U-AAC, NA 1.4, Olympus) and detected by a photodiode (S3994-01, Hamamatsu). The fluorescence signal was collected backward with a photomultiplier tube (H7422P-40, Hamamatsu) after an optical filter (715/60, Chroma). Pixel dwell time was 4 μs for each image.

### Example 13

### Safety Analysis of Curcumin-Loaded FA-GC Nanoparticles in Rats

Acute and chronic toxicity of the nanoparticles administrated to Long-Evans rats were evaluated by blood and histological analyses. The animals were randomized into a nanoparticle-treated group (n=3) or a saline-treated group (n=3). Each animal received either 1.0 ml saline containing 5.0 mg curcumin-loaded FA-GC nanoparticles or 1.0 ml pure saline through jugular vein injection. After the treatment, blood samples were collected through the jugular vein at day 1 for acute toxicity analysis, and at day 28 for chronic toxicity analysis.

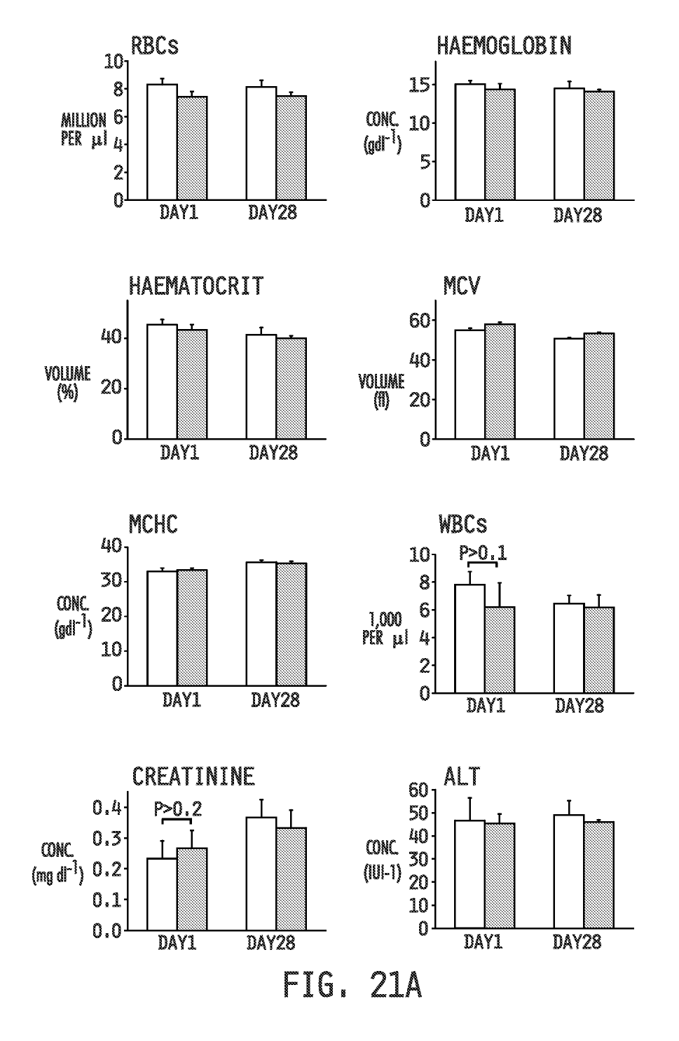

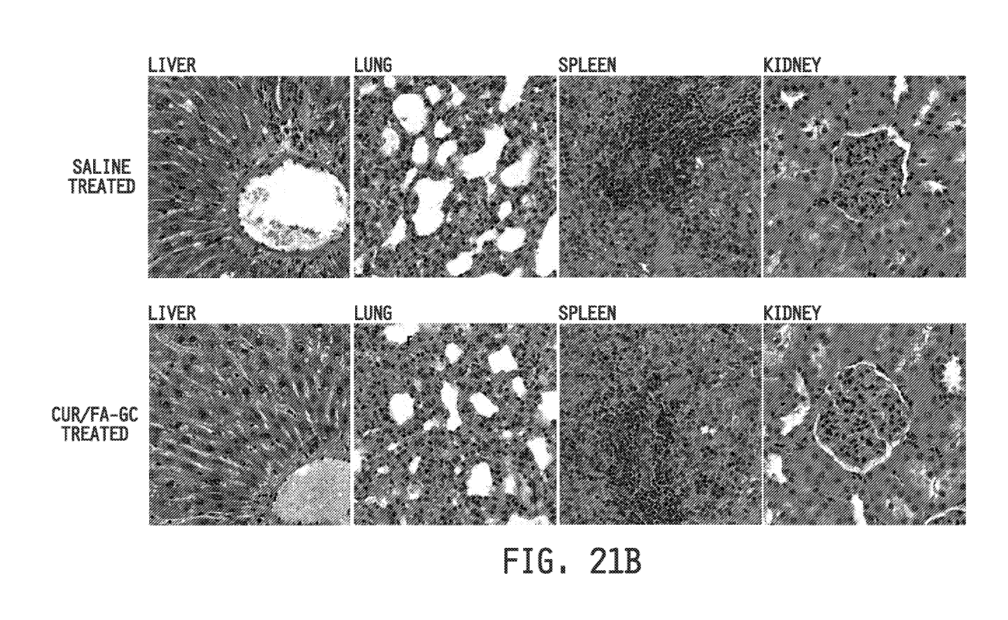

The results are shown in FIG. 21. Blood counts did not differ significantly between the two groups. In particular, the levels of creatinine and alanine transaminase (ALT) in the nanoparticle group were at the same level as those in the saline group, indicating no damage to the kidneys and the liver.

By examination of tissue morphology, the toxicity of the nanoparticles to main organs was assessed. Organs were harvested at 28 days post the treatment. No morphological difference was observed between the two groups (see FIG. 21). Together, these results suggest no adverse effects in healthy animals after systemic administration of curcumin-loaded FA-GC nanoparticles.

### Example 14

### Long Term Safety and Efficacy Analysis of Nanoparticles or Nanostructures

A long term safety and efficacy study can be performed using any of the nanoparticle or nanostructure embodiments described herein. For example, the long term study can evaluate the safety and efficacy of the hydrophobically modified nanoparticle, the polymeric nanostructure, or the polysaccharide nanoparticle over a period of one month, over a period of two months, or over a longer period of time.

Furthermore, the hydrophobically modified nanoparticle, the polymeric nanostructure, or the polysaccharide nanoparticle can be evaluated with or without addition of an anti-inflammatory agent (e.g., curcumin or a corticosteroid such as methylprednisolone).

The safety and efficacy of the nanoparticle or nanostructure can be evaluated at various timepoints over the duration of the study. For example, the safety and efficacy evaluation can take place on a daily, weekly, or monthly basis.

The safety and efficacy evaluations can include any of the parameters evaluated in the previous examples, for example the BBB scale and the toxicity parameters described herein.

1.-20. (canceled)

21. A composition comprising a hydrophobically modified nanoparticle comprising a polysaccharide and a pharmacophore, wherein the polysaccharide is covalently bound to the pharmacophore, wherein the polysaccharide is glycol chitosan and the pharmacophore is ferulic acid.

22. The composition of claim 1 further comprising a therapeutically effective amount of an anti-inflammatory agent.

23. The composition of claim 2 wherein the anti-inflammatory agent is a corticosteroid.

24. The composition of claim 3 wherein the corticosteroid is selected from the group consisting of betamethasone, dexamethasone, flumethasone, methylprednisolone, paramethasone, prednisolone, prednisone, triamcinolone, hydrocortisone, and cortisone.

25. The composition of claim 1 wherein the average diameter of the nanoparticle is about 200 to about 400 nanometers (nm).

26. A method of treating a patient having a neuronal injury, the method comprising the step of administering to the patient a therapeutically effective amount of the hydrophobically modified nanoparticle of claim 1, wherein the administration is an injection, and wherein the injection is selected from the group consisting of intraarticular, intravenous, intramuscular, intradermal, intraperitoneal, and subcutaneous injections.

27. The method of claim 6 wherein the neuronal injury is an acute neuronal injury.

28. The method of claim 6 wherein the neuronal injury is a traumatic brain injury.

29. The method of claim 6 wherein the administration is performed within 24 hours of occurrence of the neuronal injury.

30. The method of claim 6 wherein the administration is performed between about 1 hour to about 12 hours of occurrence of the neuronal injury.

31. The method of claim 6 wherein the administration is an intravenous injection.

32. The method of claim 11 wherein the administration is performed as a single dose administration.

33. A pharmaceutical formulation comprising the hydrophobically modified nanoparticle of claim 1 and a pharmaceutically acceptable carrier.

34. The pharmaceutical formulation of claim 13, wherein the carrier is saline.

35. The pharmaceutical formulation of claim 13, wherein the carrier is saline, wherein the average diameter of the nanoparticle is about 200 to about 400 nanometers (nm), and wherein the pharmaceutical formulation is formulated for intravenous administration for administration performed within 24 hours of occurrence of a neuronal injury.
